(19) United States
(12) Patent Application Publication
(10) Pub. No.: US 2025/0268944 A1
(43) Pub. Date: Aug. 28, 2025

(54) STROMAL STEM CELL THERAPEUTICS AND METHODS OF USE

(71) Applicant: Orbsen Therapeutics Limited, Galway (IE)

(72) Inventors: Larry A. COUTURE, Claremont, CA (US); Stephen J. ELLIMAN, Rosscahil (IE); Lisa O'FLYNN, Galway (IE); Jack KAVANAUGH, Los Angeles, CA (US)

(21) Appl. No.: 19/205,353

(22) Filed: May 12, 2025

Related U.S. Application Data
(63) Continuation of application No. 16/614,278, filed on Nov. 15, 2019.
(60) Provisional application No. 62/512,602, filed on May 30, 2017.

(51) Int. Cl. A61K 35/28; A61K 31/4402; A61K 31/7088; A61L 15/44; A61P 17/02

(52) U.S. Cl. CPC A61K 35/28; A61K 31/4402; A61K 31/7088; A61L 15/44; A61P 17/02; A61L 2300/64

(57) ABSTRACT

Provided herein are stromal stem cell therapeutics and methods of use in treating disease.

### CROSS REFERENCE

This application is a continuation application of U.S. application Ser. No. 16/614,278, filed Nov. 15, 2019, which is a U.S. National Stage of International Application No. PCT/IB2018/000687, filed May 30, 2018, which claims the benefit of U.S. Provisional Patent Application No. 62/512,602, filed May 30, 2017, each of which is hereby incorporated by reference in its entirety.

### BACKGROUND OF THE INVENTION

Management of chronic diseases poses challenges in a variety of medical specialties, such as liver disease and wounds. While standard treatment can often prevent or delay morbidity and mortality associated with these diseases, in many cases, individuals with inflammatory liver disease must receive a liver transplant to survive and individuals with wounds must have limbs amputated. Alternative treatments not requiring extensive surgery.

### SUMMARY OF THE INVENTION

Provided herein are methods of ameliorating at least one symptom of a wound in an individual in need thereof. Some such methods comprise topically administering to the wound a composition comprising at least 10\hat{ }3 stromal stem cells. Often, the composition comprises an extracellular matrix component. In some cases, the extracellular matrix component is a collagen. Often, the collagen is a collagen matrix. In some cases, the stromal stem cells are suspended in the collagen matrix. Often, the composition comprises at least 10\hat{ }4 stromal stem cells. Often, the composition comprises at least 10\hat{ }5 stromal stem cells. Often, the composition comprises at least 10\hat{ }6 stromal stem cells. Often, the composition comprises at least 10\hat{ }7 stromal stem cells. In some cases, the stromal stem cells are at least 30% SDC2+. In some cases, the stromal stem cells are at least 50% SDC2+. In some cases, the stromal stem cells are at least 70% SDC2+. Sometimes, the composition is a salve or an ointment. Often, the composition is affixed to a backing. In some cases, the method comprises applying a dressing to the composition and the wound. In some cases, the method comprises selecting an individual for treatment. Often, the individual is selected for having a diabetes comprising at least one of type 1 and type 2. In some cases, the selected individual is selected for having an HbA1c of 42 to 47 mmol/mol, 42 to 53 mmol/mol, 42 to 64 mmol/mol, 42 to 75 mmol/mol, 42 to 86 mmol/mol, 42 to 97 mmol/mol, 42 to 108 mmol/mol, 48 to 53 mmol/mol, 48 to 64 mmol/mol, 48 to 75 mmol/mol, 48 to 86 mmol/mol, 48 to 97 mmol/mol, 48 to 108 mmol/mol, 53 to 64 mmol/mol, 53 to 75 mmol/mol, 53 to 86 mmol/mol, 53 to 97 mmol/mol, 53 to 108 mmol/mol, 64 to 75 mmol/mol, 64 to 86 mmol/mol, 64 to 97 mmol/mol, 64 to 108 mmol/mol, 75 to 86 mmol/mol, 75 to 97 mmol/mol, 75 to 108 mmol/mol, 86 to 97 mmol/mol, 86 to 108 mmol/mol, or 97 to 108 mmol/mol. In some cases, the selected individual is selected for having an HbA1e of not more than 48 mmol/mol, 50 mmol/mol, 55 mmol/mol, 60 mmol/mol, 65 mmol/mol, 70 mmol/mol, 75 mmol/mol, 80 mmol/mol, 85 mmol/mol, 90 mmol/mol, 95 mmol/mol, 97 mmol/mol, 100 mmol/mol, or 105 mmol/mol. For example, the selected individual is selected for having an HbA1c of not more than 97 mmol/mol. In some cases, the individual is selected for having a wound that has persisted for at least 1-2 weeks, 1-4 weeks, 1-6 weeks, 1-8 weeks, 1-10 weeks, 1-12 weeks, 2-4 weeks, 2-6 weeks, 2-8 weeks, 2-10 weeks, 2-12 weeks, 4-6 weeks, 4-8 weeks, 4-10 weeks, 4-12 weeks, 6-8 weeks, 6-8 weeks, 6-10 weeks, 6-12 weeks, 8-10 weeks, 8-12 weeks, or 10-12 weeks. Often, the individual is selected for having a wound that has persisted for at least 1, 2, 4, 6, 8, 10, or 12 weeks. For example, the individual is selected for having a wound that has persisted for at least 4 weeks. Sometimes, the individual is selected for having a wound that has persisted for not more than six months, eight months, ten months, one year 1.5 years, or 2 years. For example, the individual is selected for having a wound that has persisted for not more than one year. In some cases, the individual is selected for having received standard care comprising off-loading, weekly debridement, dressings, or orthotic which have reduced the wound size by not more than 10%, 20%, 30%, 40%, 50%, 60%, 70%, 80%, or 90%. For example, the individual is selected for having received standard care which has reduced the wound size by not more than 50%. Often, the individual is selected for having a wound area of at least 0.1 cm\hat{ }2, 0.2 cm\hat{ }2, 0.3 cm\hat{ }2, 0.4 cm\hat{ }2, 0.5 cm\hat{ }2, 0.6 cm\hat{ }2, 0.7 cm\hat{ }2, 0.8 cm\hat{ }2, 0.9 cm\hat{ }2, 1.0 cm\hat{ }2, 1.5 cm\hat{ }2, 2.0 cm\hat{ }2, 2.5 cm\hat{ }2, 3.0 cm\hat{ }2, 3.5 cm\hat{ }2, 4.0 cm\hat{ }2, 4.5 cm\hat{ }2, or 5.0 cm\hat{ }2. For example, the individual is selected for having a wound area of at least 0.5 cm\hat{ }2. In some cases, the individual is selected for having a wound area of not more than 1.0 cm\hat{ }2, 1.5 cm\hat{ }2, 2.0 cm\hat{ }2, 2.5 cm\hat{ }2, 3.0 cm\hat{ }2, 3.5 cm\hat{ }2, 4.0 cm\hat{ }2, 4.5 cm\hat{ }2, or 5.0 cm\hat{ }2. For example, the individual is selected for having a wound area of not more than 4.0 cm\hat{ }2. Often, the individual is selected for having a wound having a Texas wound stage comprising 1a, 1c, or 2a. In some cases, the individual is selected for having a wound on the leg, ankle, or foot. In some cases, the individual is selected for having a wound located distal to the malleolus. In some cases, the individual is selected for having a pressure in a toe of an affected limb is at least 20 mmHg, 30 mmHg, 40 mmHg, 50 mmHg, or 60 mmHg. For example, the individual is selected for having a pressure in a toe of an affected limb is at least 40 mmHg. Often, the individual is selected for having an ankle-brachial systolic pressure index is from about 0.5 to about 1.5, about 0.5 to about 1.4, about 0.5 to about 1.3, about 0.5 to about 1.2, about 0.5 to about 1.1, about 0.6 to about 1.5, about 0.6 to about 1.4, about 0.6 to about 1.3, about 0.6 to about 1.2, about 0.6 to about 1.1, about 0.7 to about 1.5, about 0.7 to about 1.4, about 0.7 to about 1.3, about 0.7 to about 1.2, about 0.7 to about 1.1, about 0.8 to about 1.5, about 0.8 to about 1.4, about 0.8 to about 1.3, about 0.8 to about 1.2, about 0.8 to about 1.1, about 0.9 to about 1.5, about 0.9 to about 1.4, about 0.9 to about 1.3, about 0.9 to about 1.2, or about 0.9 to about 1.1. For example, the individual is selected for having an ankle-brachial systolic pressure index is from about 0.7 to about 1.3. Sometimes, the individual is selected for having a diagnosis of peripheral neuropathy, for example a diagnosis of peripheral neuropathy using ADA guidelines. Often, the wound is fully closed after a single administration. In some cases, the wound is fully closed after a second administration of the composition. Often, the wound is an open wound, a non-self-healing wound, a dermal wound, or an ulcerative wound.

Also provided herein are methods of preparing a wound dressing for a wound, such as a diabetic wound. Some such methods comprise: (a) obtaining a first composition comprising stromal stem cells; (b) obtaining a second composition comprising collagen; (c) preparing a mixture of the first composition and the second composition; and (d) applying the resulting mixture to a backing having an adhesive. In some cases, the first composition comprises at least 10\hat{ }2, 10\hat{ }3, 10\hat{ }4, 10\hat{ }5, or 10\hat{ }6 stromal stem cells. Often, the second composition comprises at least 3%, 4%, 5%, 6%, 7%, 8%, 9%, or 10% collagen. In some cases, some such methods comprise: (a) obtaining a first composition comprising at least 10\hat{ }3 stromal stem cells; (b) obtaining a second composition comprising at least 6% collagen; (c) preparing a mixture of the first composition and the second composition; and (d) applying the resulting mixture to a backing having an adhesive for applying to the diabetic wound. In some cases, the resulting mixture comprises at least 10\hat{ }3 stromal stem cells and 2.6% collagen. Often, the resulting mixture comprises a collagen matrix. Often, the mixing comprises passing each component between two luer lok syringes. In some cases, the stromal stem cells are at least 30% SDC2+. Often, the stromal stem cells are at least 50% SDC2+. In some cases, the stromal stem cells are at least 70% SDC2+.

Also provided herein are compositions comprising stromal stem cells; an extracellular matrix; and a backing. In some cases, the first composition comprises at least 10\hat{ }2, 10\hat{ }3, 10\hat{ }4, 10\hat{ }5, or 10\hat{ }6 stromal stem cells. In some cases, the composition comprises at least 10\hat{ }3 stromal stem cells. Often, the extracellular matrix comprises collagen or hyaluronic acid. In some cases, the composition comprises at least 1%, 2%, 3%, 4%, 5%, 6%, 7%, 8%, 9%, or 10% collagen. In some cases, the composition comprises at least 1%, 2%, 3%, 4%, 5%, 6%, 7%, 8%, 9%, or 10% hyaluronic acid. In some cases, the composition comprises at least 2% collagen. Often, the backing comprises an adhesive. Often, the stromal stem cells are at least 30% SDC2+. In some cases, the stromal stem cells are at least 50% SDC2+. Often, the stromal stem cells are at least 70% SDC2+. In some cases, the stromal stem cells are suspended in the extracellular matrix.

Further provided herein are methods of reducing at least one symptom of a liver disease in an individual in need thereof. In some cases, methods herein reduce at least one symptom of an inflammatory liver disease. Some such methods comprise administering a composition comprising at least 10\hat{ }3 stromal stem cells/kg to the individual. In some cases, the stromal stem cells are at least 30% SDC2+. Often, the stromal stem cells are at least 50% SDC2+. In some cases, the stromal stem cells are at least 70% SDC2+. Often, the composition comprises at least 10\hat{ }4 stromal stem cells/kg. Often, the composition comprises at least 10\hat{ }5 stromal stem cells/kg. Often, the composition comprises at least 10\hat{ }6 stromal stem cells/kg. In some cases, the composition comprises at least 1.0×10\hat{ }6 stromal stem cells/kg. In some cases, the composition comprises at least 2.5×10\hat{ }6 stromal stem cells/kg. Often, the composition is administered intravenously. In some cases, the method comprises selecting an individual having an inflammatory liver disease. Sometimes, the individual is selected for having an inflammatory liver disease selected from autoimmune hepatitis and primary sclerosing cholangitis. Often, the individual is selected for having a serum alkaline phosphatase (ALP) of at least 1.5 ULN. In some cases, the individual is selected for having a serum alanine aminotransferase of at least 1.5 ULN. Sometimes, the individual is selected for having standard of care treatment for the inflammatory liver disease for at least 24 weeks prior to treatment. In some cases, the method comprises administration of an immunosuppressant. Often, the method comprises administration of chlorpheniramine. In some cases, the individual shows improvement in at least one measure of liver function selected from alkaline phosphatase, alanine transaminase, aspartate transaminase, albumin, bilirubin, gamma glutamyltransferase, total bile acid, immunoglobulin, and C-reactive protein four weeks after treatment. Sometimes, the individual shows reduced liver fibrosis four weeks after treatment. Often, the individual shows a reduction in fatigue four weeks after treatment. In some cases, the individual does not require a liver transplant for at least one year following treatment. Often, the individual shows improvement in at least one of osteoporosis, serum cholesterol, xanthomas, absorption of fat soluble vitamins, edema, ascites, hepatic encephalopathy, hypersplenism, hypothyroidism, sicca syndrome, Raynaud's phenomenon, scleroderma, ciliac sprue, urinary tract infections, gallstones, jaundice, fatigue, dark urine, pale stool, pain, loss of appetite, and weight loss four weeks after treatment. Sometimes, the individual maintains healthy liver function for at least 6 months after treatment. Often, the individual maintains healthy liver function for at least 12 months after treatment.

### BRIEF DESCRIPTION OF THE DRAWINGS

An understanding of the features and advantages of the present invention will be obtained by reference to the following detailed description that sets forth illustrative embodiments, in which the principles of the invention are utilized, and the accompanying drawings of which:

FIG. 1 shows the number of individuals needing a liver transplant each year due to illness with primary biliary cholangitis (PBC), primary sclerosing cholangitis (PSC), and autoimmune hepatitis (AIH).

FIG. 2 shows proliferation of CD362+ (SDC2+) mesenchymal stem cells compared with bone marrow derived mesenchymal stem cells.

FIG. 3A shows reduced serum ALT in MDR2KO/FVB mice treated with CD362+ (SDC2+) mesenchymal stem cells.

FIG. 3B shows reduced serum ALT in Ova-bil mice treated with CD362+ (SDC2+) mesenchymal stem cells.

FIG. 3C shows reduced serum ALT in acute CCL4 mice treated with CD362+ (SDC2+) mesenchymal stem cells.

FIG. 4A shows reduction in histological inflammation in MDR2KO/FVB mice treated with CD362+ (SDC2+) mesenchymal stem cells.

FIG. 4B shows reduction in histological liver injury in MDR2KO/FVB mice treated with CD362+ (SDC2+) mesenchymal stem cells.

FIG. 5A shows reduction in hepatic CD3+ T cells in mice treated with CD362+ (SDC2+) mesenchymal stem cells.

FIG. 5B shows reduction in hepatic CD4+ T cells in mice treated with CD362+ (SDC2+) mesenchymal stem cells.

FIG. 5C shows reduction in hepatic CD8+ T cells in mice treated with CD362+ (SDC2+) mesenchymal stem cells.

FIG. 6 shows increase in regulatory T cells in mice treated with CD362+ (SDC2+) mesenchymal stem cells.

FIG. 7 shows reduction in liver macrophage M2 polarization in mice treated with CD362+ (SDC2+) mesenchymal stem cells.

FIG. 8 shows reduction in T cell proliferation in healthy volunteer CD4+ T cells treated with CD362+ (SDC2+) mensenchymal stem cells.

FIG. 9 shows reduction in T cell proliferation in PSC volunteer CD4+ T cells treated with CD362+ (SDC2+) mensenchymal stem cells.

FIG. 10A shows reduction in CD8+ T cell lysis in CD8+ T cells treated with CD362+ (SDC2+) mensenchymal stem cells.

FIG. 10B shows reduction in CD8+ T cell lysis in CD8+ T cells treated with CD362+ (SDC2+) mensenchymal stem cells.

FIG. 11 shows reduction in CD4+ T cell proliferation in cells treated with CD362+ (SDC2+) mensenchymal stem cells treated with IFN-gamma.

FIG. 12 shows increase in arterial oxygen FI0.3 (UC) in Sprague Dawley rats with lung injury treated with CD362+ (SDC2+) mensenchymal stem cells.

FIG. 13A shows decrease in TNF-alpha induced ICAM-1 in endothelial cells incubated with mesenchymal stem cells.

FIG. 13B shows decrease in TNF-alpha induced VCAM-1 in endothelial cells incubated with mesenchymal stem cells.

FIG. 14 shows preparation and administration of stromal stem cells for wound treatment.

FIG. 15A shows a representative gating strategy for purification of CD362 (SDC2) stromal stem cells.

FIG. 15B shows identification of viable CD362 (SDC2) stromal stem cells.

FIG. 16 shows average percentage population of CD362+ stromal stem cells at various steps of the purification process.

FIG. 17A shows average fold enrichment of CD362+ stromal stem cell after each sort.

FIG. 17B shows average percentage recovery of CD362+ stromal stem cells after each sort.

FIG. 18 shows CFU-f analysis of colonies formed after sort.

### DETAILED DESCRIPTION OF THE INVENTION

Stromal stem cells or mesenchymal stem cells disclosed herein are purified or isolated from a population of cells based on expression of the cell surface marker SDC2. Stromal stem cells are population of immunomodulatory fibroblastic cells that are isolated from one or more of human bone marrow, adipose tissue, placenta and umbilical cord tissue. In some cases, small numbers of stromal stem cells are isolated from these tissues and cultured in vitro to proliferate as plastic-adherent cells, to form colonies of fibroblasts (CFU-F). Stromal stem cells often act as immune system modulators. Stromal stem cells, in some cases, also secrete proteins and extracellular vesicles (exosomes) that contain significant immuno-suppressive factors such as transforming growth factor β1 (TGFβ1), Indoleamine 2,3-dioxygenase 1(IDO1), TNF-stimulated gene 6 (TSG6) and the purinergic enzymes CD39 and CD73. Using this collection of factors, stromal stem cells induce numbers of regulatory T cells, suppress proliferation of both T helper and cytotoxic T cells, decrease the production of the pro-inflammatory cytokines interferon γ (IFN-γ), tumor necrosis factor α (TNF-α) and IL-2, inhibit the activation of natural killer cells, arrest B-cell maturation, and block maturation of dendritic cells, resulting in reduced expression of antigens and co-stimulatory molecules necessary to activate T-cells. Accordingly, disclosed herein are methods of treatment or symptom amelioration using allogeneic stromal stem cells immunotherapy, for the treatment of inflammatory diseases including but not limited to liver diseases, such as inflammatory liver diseases, and wounds, such as non-healing wounds and diabetic wounds.

### Methods of Treating Inflammatory Liver Disease

Disclosed herein are methods of treating a liver disease in an individual in need thereof such as inflammatory liver disease. Methods of reducing at least one symptom of an inflammatory liver disease in an individual in need thereof comprise administering a composition comprising stromal stem cells to the individual. Liver diseases, such as inflammatory liver diseases herein include but are not limited to autoimmune hepatitis, primary biliary cirrhosis, and primary sclerosing cholangitis. These diseases contribute disproportionately to the morbidity and mortality of chronic liver disease, which accounts for 1.8% of all deaths and nearly 20% of all transplants in Europe. Symptoms of inflammatory liver disease include reduced liver function as measured by alkaline phosphatase, alanine transaminase, aspartate transaminase, albumin, bilirubin, gamma glutamyltransferase, total bile acid, immunoglobulin, and/or C-reactive protein; liver fibrosis; fatigue; osteoporosis; aberrant serum cholesterol; xanthomas; aberrant absorption of fat soluble vitamins; edema; ascites; hepatic encephalopathy; hypersplenism; hypothyroidism; sicca syndrome; Raynaud's phenomenon; scleroderma; ciliac sprue; urinary tract infections; gallstones; jaundice; dark urine; pale stool; pain; loss of appetite; and weight loss.

### Patient Selection

Individuals selected for treatment of inflammatory liver diseases based on suitability for the treatment methods provided herein. In some cases, individuals selected for treatment are at least 18 years old or no more than 70 years old. Alternatively, individuals selected for treatment are 18 to 20 years old, 18 to 25 years old, 18 to 30 years old, 18 to 35 years old, 18 to 40 years old, 18 to 45 years old, 18 to 50 years old, 18 to 55 years old, 18 to 60 years old, 18 to 65 years old, 18 to 70 years old, 20 to 25 years old, 20 to 30 years old, 20 to 35 years old, 20 to 40 years old, 20 to 45 years old, 20 to 50 years old, 20 to 55 years old, 20 to 60 years old, 20 to 65 years old, 20 to 70 years old, 25 to 30 years old, 25 to 35 years old, 25 to 40 years old, 25 to 45 years old, 25 to 50 years old, 25 to 55 years old, 25 to 60 years old, 25 to 65 years old, 25 to 70 years old, 30 to 35 years old, 30 to 40 years old, 30 to 45 years old, 30 to 50 years old, 30 to 55 years old, 30 to 60 years old, 30 to 65 years old, 30 to 70 years old, 35 to 40 years old, 35 to 45 years old, 35 to 50 years old, 35 to 55 years old, 35 to 60 years old, 35 to 65 years old, 35 to 70 years old, 40 to 45 years old, 40 to 50 years old, 40 to 55 years old, 40 to 60 years old, 40 to 65 years old, 40 to 70 years old, 45 to 50 years old, 45 to 55 years old, 45 to 60 years old, 45 to 65 years old, 45 to 70 years old, 50 to 55 years old, 50 to 60 years old, 50 to 65 years old, 50 to 70 years old, 55 to 60 years old, 55 to 65 years old, 55 to 70 years old, 60 to 65 years old, 60 to 70 years old, or 65 to 70 years old.

In methods herein, individuals selected for treatment are selected, in some cases, for having primary sclerosing cholangitis (PSC). Some such individuals are often observed to have chronic biochemical cholestasis, such as elevated serum alkaline phosphatase (ALP) or elevated gama-glutamyl transpeptidase (GGT) which are observed to be above the upper limit of normal for at least six months. Individuals are sometimes observed to have an ALP level at least 1.5 times the upper limit of normal. In some cases, individuals are observed to have an ALP level at least 1.5, 2.0, 2.5, 3.0, 4.0, 5.0, 6.0, 7.0, 8.0, 9.0 or more times the upper limit of normal. Alternatively, individuals are observed to have an ALP level no more than 10 times the upper limit of normal. In some cases, ALP and GGT are observed to be above the upper limit of normal for at least 2 months, 3 months, 4 months, 5 months, 6 months, 7 months, 8 months, 9 months, 10 months, 11 months, 12 months, or longer. In some cases, individuals are observed to have radiological or histological evidence of PSC. In some cases, individuals are observed to have no documented alternative etiology for sclerosing cholangitis (i.e. secondary sclerosing cholangitis). In some cases, individuals are observed to have no evidence of dominant alternative chronic or active liver injury other than PSC. In some cases, individuals are observed to have no evidence of cholangitis. In some cases, individuals are observed to have no need for any antibiotics. In some cases, individuals are observed to have no presence of percutaneous biliary drain, or internal biliary stent. In some cases, individuals are observed to have no diagnosed hepatocellular carcinoma, cholangiocarcinoma. Often individuals are observed to have no dominant stricture clinically suspicious of cholangiocarcinoma.

Additionally, in methods herein, individuals are selected for treatment in some cases, for having autoimmune hepatitis (AIH). Suitable criteria for diagnosis with autoimmune hepatitis are consistent with the simplified Autoimmune Hepatitis Group (IAIHG) criteria. In some cases, individuals are observed to have histological evidence of AIH in a liver biopsy. Individuals are sometimes observed to have an alanine transaminase (ALT) level at least 1.5 times the upper limit of normal. In some cases, individuals are observed to have an ALT level at least 1.5, 2.0, 2.5, 3.0, 4.0, 5.0, 6.0, 7.0, 8.0, 9.0 or more times the upper limit of normal. In some cases, individuals are selected for having AST or ALT levels of no more than 10 times the upper limit of normal. Often, individuals are selected for having standard AIH treatment, excluding biologics for at least 24 weeks. Alternatively, individuals are selected for having standard AIH treatment for at least 10 weeks, 12 weeks, 14 weeks, 16 weeks, 18 weeks, 20 weeks, 22 weeks, 24 weeks, 26 weeks, 28 weeks, 30weeks, 32 weeks, 48 weeks, 60 weeks, or more. In some cases, individuals have been given stable doses of immunosuppression for at least 4 weeks prior to treatment. Alternatively, individuals have been given stable doses of immunosuppression for at last 2 weeks, 4 weeks, 6 weeks, 8 weeks, 10 weeks, 12 weeks, 14 weeks, 16 weeks, 18 weeks, or more. In some cases, it is desirable that individuals continue immunosuppression throughout the treatment with stromal stem cells provided herein. In some cases, individuals are observed to have no treatment with prednisolone with a dose of >20 mg. In some cases, individuals are observed to have no diagnosed hepatocellular carcinoma or cholangiocarcinoma.

Individuals, in some cases, are selected for treatment when it is observed that creatinine is less than 133 μmol/L. In some cases, individuals are selected for treatment when they have not been treated with renal replacement therapy. Often individuals are selected for treatment when they are observed to have aspartate aminotransferase (AST) of less than 10 times the upper limit of normal. In some cases, individuals are selected for treatment when they are observed to have ALT of less than 10 times the upper limit of normal. Sometimes, individuals are selected for treatment when they are observed to have ALP of less than 10 times the upper limit of normal. Often individuals are selected for treatment when they are observed to have platelet levels of less than 50×10\hat{ }9/L. In some cases, individuals are selected for treatment when they are observed to have total bilirubin levels of less than 2 times the upper limit of normal. Often, individuals are selected for treatment when they are observed to have international normalized ratio of less than 1.3 in the absence of anti-coagulant therapy. In some cases, individuals are selected for treatment when they are observed to have albumin levels greater than 35 g/liter. Often, individuals are selected for treatment when they are observed to have hemoglobin levels of greater than 10 g/dl. In some cases, individuals are selected for treatment when they are observed to have no past or present evidence of decompensated chronic liver disease. Often, individuals are selected for treatment when they are observed to have no radiological or clinical evidence of ascites. In some cases, individuals are selected for treatment when they are observed to have no signs of hepatic encephalopathy. In some cases, individuals are selected for treatment when they are observed to have no endoscopic evidence of portal hypertensive bleeding.

Some compositions for treating inflammatory liver disease disclosed herein comprise stromal stem cells wherein the population of stromal stem cells in the composition is at least 30% SDC2+. In some cases, the population of stromal stem cells in the composition is at least 40% SDC2+. In some cases, the population of stromal stem cells in the composition is at least 50% SDC2+. In some cases, the population of stromal stem cells in the composition is at least 60% SDC2+. Often, the population of stromal stem cells in the composition is at least 70% SDC2+. In some cases, the population of stromal stem cells in the composition is at least 80% SDC2+. Sometimes, the population of stromal stem cells in the composition is at least 90% SDC2+. Often, the population of stromal stem cells in the composition is at least 95% SDC2+. Sometimes, the population of stromal stem cells in the composition is at least 97% SDC2+. In some cases, the population of stromal stem cells in the composition is at least 99% SDC2+. In some cases, the population of stromal stem cells in the composition is substantially 100% SDC2+.

Compositions for treating inflammatory liver disease are provided in therapeutically effective doses, in some cases, metered based on body weight of the individual treated. Often, the composition comprises at least 10\hat{ }4 stromal stem cells/kg. In some cases, the composition comprises at least 10\hat{ }5 stromal stem cells/kg. Sometimes, the composition comprises at least 10\hat{ }6 stromal stem cells/kg. Sometimes, the composition comprises at least 1.0×10\hat{ }6 stromal stem cells/kg. In some cases, the composition comprises at least 2.5×10\hat{ }6 stromal stem cells/kg. In some cases, the composition comprises 1×10\hat{ }5 stromal stem cells. In some cases, the composition comprises 5×10\hat{ }5 stromal stem cells. Sometimes, the composition comprises 1×10\hat{ }6 stromal stem cells. Often, the composition comprises 2×10\hat{ }6 stromal stem cells. Sometimes, the composition comprises 3×10\hat{ }6 stromal stem cells. Sometimes, the composition comprises 4×10\hat{ }6 stromal stem cells. Often, the composition comprises 5×10\hat{ }6 stromal stem cells. In some cases, the composition comprises 6×10\hat{ }6 stromal stem cells. In some cases, the composition comprises 7×10\hat{ }6 stromal stem cells. In some cases, the composition comprises 8×10\hat{ }6 stromal stem cells. Sometimes, the composition comprises 9×10\hat{ }6 stromal stem cells. In some cases, the composition comprises 1×10\hat{ }7 stromal stem cells. In some cases, the composition comprises 2×10\hat{ }7 stromal stem cells. Sometimes, the composition comprises 3×10\hat{ }7 stromal stem cells. Often, the composition comprises 4×10\hat{ }7 stromal stem cells. In some cases, the composition comprises 5×10\hat{ }7 stromal stem cells. Often, the composition comprises 6×10\hat{ }7 stromal stem cells. Often, the composition comprises 7×10\hat{ }7 stromal stem cells. Sometimes, the composition comprises 8×10\hat{ }7 stromal stem cells. In some cases, the composition comprises 9×10\hat{ }7 stromal stem cells. In some cases, the composition comprises 1.0×10\hat{ }8 stromal stem cells. In some cases, the composition comprises 1.2×10\hat{ }8 stromal stem cells. In some cases, the composition comprises 1.4×10\hat{ }8 stromal stem cells. Often, the composition comprises 1.6×10\hat{ }8 stromal stem cells. Sometimes, the composition comprises 1.8×10\hat{ }8 stromal stem cells. In some cases, the composition comprises 2.0×10\hat{ }8 stromal stem cells. In some cases, the composition comprises 2.2×10\hat{ }8 stromal stem cells. Often, the composition comprises 2.4×10\hat{ }8 stromal stem cells. Often, the composition comprises 2.8×10\hat{ }8 stromal stem cells. Sometimes, the composition comprises 3.0×10\hat{ }8 stromal stem cells. In some cases, the composition comprises 3.2×10\hat{ }8 stromal stem cells. In some cases, the composition comprises 3.4×10\hat{ }8 stromal stem cells. Often, the composition comprises 3.6×10\hat{ }8 stromal stem cells. Often, the composition comprises 3.8×10\hat{ }8 stromal stem cells. Sometimes, the composition comprises 4.0×10\hat{ }8 stromal stem cells.

In some cases individuals treated using methods herein are required to fast on the day of infusion. In some cases, individuals treated using methods herein are able to cat and drink after infusion with cells has commenced. Individuals receiving treatment with methods herein are often administered chlorpheniramine as a 10 mg by peripheral IV bolus. In some cases, individuals are given approximately a 10 mg dose of chlorpheniramine 30 minutes prior to infusion.

Methods of treatment herein comprise one or more administrations of stromal stem cell compositions in doses disclosed herein. In some cases, methods comprise one administration of stromal stem cell compositions. In some cases, methods comprise two administrations of stromal stem cell compositions. In some cases, methods comprise three administrations of stromal stem cell compositions. In some cases, methods comprise four administrations of stromal stem cell compositions. In some cases, methods comprise five administrations of stromal stem cell compositions. In some cases, methods comprise six administrations of stromal stem cell compositions. In some cases, one or more administrations of stromal stem cells are administered daily. In some cases, one or more administrations of stromal stem cells are administered weekly. In some cases, one or more administrations of stromal stem cells are administered biweekly. In some cases, one or more administrations of stromal stem cells are administered monthly. In some cases, one or more administrations of stromal stem cells are administered every three months. In some cases, one or more administrations of stromal stem cells are administered every six months. In some cases, one or more administrations of stromal stem cells are administered yearly.

A number of administration routes are consistent with the disclosure herein, such as parenteral administration. Examples include but are not limited to intravenous, intra-arterial, intramuscular, intraperitoneal, and/or subcutaneous. In some cases, individuals are administered stromal stem cells via infusion via large-bore (green/18G or larger) cannula by Y-connector giving-set. In some cases, each bag of stromal stem cells is given over 10-15 minutes, and given serially via the same giving set. In some cases, the other arm of the y-connector is attached to a 250 ml bag of normal saline, which is infused over the total anticipated duration of the MSC infusion. Often, the end of the last bag of the MSC marks the end of the MSC infusion. In some cases, upon completion of the MSC infusion, the remaining cells are flushed with 250 ml of normal saline (over 30 mins) which is infused via the same arm of the y-connector as the MSC. Often, peri-infusion vital signs monitored include 5 minutes pre-infusion. In some cases, vital signs are monitored every 15 minutes throughout the infusion; then at 30, 60, 120 and 240 minutes post cessation. In some cases, standard bloods and exploratory research bloods are taken 4 hours after completion of infusion.

In some methods provided herein, methods of treatment comprise selecting an individual having an inflammatory liver disease, for example autoimmune hepatitis, primary biliary cirrhosis, or primary sclerosing cholangitis. In some cases, the individual is selected for having a serum alkaline phosphatase (ALP) of at least 1.5 ULN. In some cases, the individual is selected for having a serum alanine aminotransferase of at least 1.5 ULN. Sometimes, the individual is selected for having chronic biochemical cholestasis. In some cases the individual is selected for having radiological and/or histological evidence of clinically documented primary sclerosing cholangitis. Often, the individual is selected for having a diagnosis of autoimmune hepatitis defined by IAIHG criteria. In some cases, the individual is selected for having autoimmune hepatitis as diagnosed by liver biopsy. In some cases, the individual is selected for having standard of care treatment for the inflammatory liver disease for at least 24 weeks prior to treatment.

Methods of treatment herein, in some cases, comprise administration of one or more additional therapeutics before, during, and/or after administration of stromal stem cell compositions. Often, the method comprises administration of an immunosuppressant. Sometimes, the method comprises administration of chlorpheniramine. In some cases, the method comprises administration of an antihistamine, such as, afexofenadine, a terfenadine, a triprolidine, a bropheniramine, a chlorpheniramine, a cetirizine, a diphenhydramine, a carbinoxamine, a promethazine, a loratadine, or a levocetirizine. In some cases, the method comprises administration of a corticosteroid, such as a hydrocortisone, a methylprednisolone, a prednisolone, a prednisone, or a triamcinolone.

Methods of treatment herein often result in improvement in at least one symptom of inflammatory liver disease. In some cases, the individual is observed to show improvement in at least one measure of liver function selected from alkaline phosphatase, alanine transaminase, aspartate transaminase, albumin, bilirubin, gamma glutamyltransferase, total bile acid, immunoglobulin, and C-reactive protein. In some cases, the individual shows reduced liver fibrosis. In some cases, individual is observed to show a reduction in fatigue. In some cases, the individual is observed to not require a liver transplant. In some cases, the individual is observed to show improvement in at least one of osteoporosis, serum cholesterol, xanthomas, absorption of fat soluble vitamins, edema, ascites, hepatic encephalopathy, hypersplenism, hypothyroidism, sicca syndrome, Raynaud's phenomenon, scleroderma, ciliac sprue, urinary tract infections, gallstones, jaundice, fatigue, dark urine, pale stool, pain, loss of appetite, and weight loss. In some cases, the individual is observed to maintain healthy liver function after treatment.

Methods of treatment herein result in observation of improvement in one or more symptoms after receiving stromal stem cell compositions herein. In some cases, individuals are observed to improve within one week of receiving stromal stem cell compositions. In some cases, individuals are observed to improve within two weeks of receiving stromal stem cell compositions. In some cases, individuals are observed to improve within three weeks of receiving stromal stem cell compositions. In some cases, individuals are observed to improve within four week of receiving stromal stem cell compositions. In some cases, individuals are observed to improve within one month of receiving stromal stem cell compositions. In some cases, individuals are observed to improve within two months of receiving stromal stem cell compositions. In some cases, individuals are observed to maintain healthy liver function for at least two months after receiving stromal stem cell compositions. In some cases, individuals are observed to maintain healthy liver function for at least three months after receiving stromal stem cell compositions. In some cases, individuals are observed to maintain healthy liver function for at least four months after receiving stromal stem cell compositions. In some cases, individuals are observed to maintain healthy liver function for at least five months after receiving stromal stem cell compositions. In some cases, individuals are observed to maintain healthy liver function for at least six months after receiving stromal stem cell compositions. In some cases, individuals are observed to maintain healthy liver function for at least eight months after receiving stromal stem cell compositions. In some cases, individuals are observed to maintain healthy liver function for at least ten months after receiving stromal stem cell compositions. In some cases, individuals are observed to maintain healthy liver function for at least twelve months after receiving stromal stem cell compositions.

### Methods of Treating Wounds

Disclosed herein are methods of treating wounds, such as non-healing wounds. The current standard care involves removal of pressure from the wound, restoration of blood flow if peripheral vascular disease is present, debridement of the wound, and institution of antibiotic therapy to control infection. Topical dressings, patient education, podiatry review, and orthotics are part of standard care. It has been reported that for standard treatment of neuropathic diabetic ulcers, where blood supply had been adequate (as defined by a transcutaneous oxygen pressure of >30 mmHg or an ankle-brachial index >0.7), after 20 weeks 31% of diabetic neuropathic ulcers were healed and at 12 weeks, 24% of neuropathic ulcers were completely healed. Methods herein provide an improvement over current standard care.

Some such methods include methods of ameliorating at least one symptom of a wound in an individual in need thereof comprising topically administering to the wound a composition comprising stromal stem cells. Individuals are selected for treatment of dermal wounds with stromal stem cells when they are at least 18 years old or no more than 70 years old, for example 18 to 20 years old, 18 to 25 years old, 18 to 30 years old, 18 to 35 years old, 18 to 40 years old, 18 to 45 years old, 18 to 50 years old, 18 to 55 years old, 18 to 60 years old, 18 to 65 years old, 18 to 70 years old, 20 to 25 years old, 20 to 30 years old, 20 to 35 years old, 20 to 40 years old, 20 to 45 years old, 20 to 50 years old, 20 to 55 years old, 20 to 60 years old, 20 to 65 years old, 20 to 70 years old, 25 to 30 years old, 25 to 35 years old, 25 to 40 years old, 25 to 45 years old, 25 to 50 years old, 25 to 55 years old, 25 to 60 years old, 25 to 65 years old, 25 to 70 years old, 30 to 35 years old, 30 to 40 years old, 30 to 45 years old, 30 to 50 years old, 30 to 55 years old, 30 to 60 years old, 30 to 65 years old, 30 to 70 years old, 35 to 40 years old, 35 to 45 years old, 35 to 50 years old, 35 to 55 years old, 35 to 60 years old, 35 to 65 years old, 35 to 70 years old, 40 to 45 years old, 40 to 50 years old, 40 to 55 years old, 40 to 60 years old, 40 to 65 years old, 40 to 70 years old, 45 to 50 years old, 45 to 55 years old, 45 to 60 years old, 45 to 65 years old, 45 to 70 years old, 50 to 55 years old, 50 to 60 years old, 50 to 65 years old, 50 to 70 years old, 55 to 60 years old, 55 to 65 years old, 55 to 70 years old, 60 to 65 years old, 60 to 70 years old, or 65 to 70 years old.

In some cases, individuals are selected for treatment when they are observed to have a diabetes, such as diabetes mellitus, including Type 1 and Type 2 diabetes mellitus. In some cases, the selected individual is selected for having an HbA1c of 42 to 47 mmol/mol, 42 to 53 mmol/mol, 42 to 64 mmol/mol, 42 to 75 mmol/mol, 42 to 86 mmol/mol, 42 to 97 mmol/mol, 42 to 108 mmol/mol, 48 to 53 mmol/mol, 48 to 64 mmol/mol, 48 to 75 mmol/mol, 48 to 86 mmol/mol, 48 to 97 mmol/mol, 48 to 108 mmol/mol, 53 to 64 mmol/mol, 53 to 75 mmol/mol, 53 to 86 mmol/mol, 53 to 97 mmol/mol, 53 to 108 mmol/mol, 64 to 75 mmol/mol, 64 to 86 mmol/mol, 64 to 97 mmol/mol, 64 to 108 mmol/mol, 75 to 86 mmol/mol, 75 to 97 mmol/mol, 75 to 108 mmol/mol, 86 to 97 mmol/mol, 86 to 108 mmol/mol, or 97 to 108 mmol/mol. In some cases, the selected individual is selected for having an HbA1c of not more than 48 mmol/mol, 50 mmol/mol, 55 mmol/mol, 60 mmol/mol, 65 mmol/mol, 70 mmol/mol, 75 mmol/mol, 80 mmol/mol, 85 mmol/mol, 90 mmol/mol, 95 mmol/mol, 97 mmol/mol, 100 mmol/mol, or 105 mmol/mol. For example, the selected individual is selected for having an HbA1c of not more than 97 mmol/mol. In some cases, the individual is selected for having a wound that has persisted for at least 1-2 weeks, 1-4 weeks, 1-6 weeks, 1-8 weeks, 1-10 weeks, 1-12 weeks, 2-4 weeks, 2-6 weeks, 2-8 weeks, 2-10 weeks, 2-12 weeks, 4-6 weeks, 4-8 weeks, 4-10 weeks, 4-12 weeks, 6-8 weeks, 6-8 weeks, 6-10 weeks, 6-12 weeks, 8-10 weeks, 8-12 weeks, or 10-12 weeks. Often, the individual is selected for having a wound that has persisted for at least 1, 2, 4, 6, 8, 10, or 12 weeks. For example, the individual is selected for having a wound that has persisted for at least 4 weeks. Sometimes, the individual is selected for having a wound that has persisted for not more than six months, eight months, ten months, one year 1.5 years, or 2 years. For example, the individual is selected for having a wound that has persisted for not more than one year. In some cases, the individual is selected for having received standard care comprising off-loading, weekly debridement, dressings, or orthotic which have reduced the wound size by not more than 10%, 20%, 30%, 40%, 50%, 60%, 70%, 80%, or 90%. For example, the individual is selected for having received standard care which has reduced the wound size by not more than 50%. Often, the individual is selected for having a wound area of at least 0.1 cm\hat{ }2, 0.2 cm\hat{ }2, 0.3 cm\hat{ }2, 0.4 cm\hat{ }2, 0.5 cm\hat{ }2, 0.6 cm\hat{ }2, 0.7 cm\hat{ }2, 0.8 cm\hat{ }2, 0.9 cm\hat{ }2, 1.0 cm\hat{ }2, 1.5 cm\hat{ }2, 2.0 cm\hat{ }2, 2.5 cm\hat{ }2, 3.0 cm\hat{ }2, 3.5 cm\hat{ }2, 4.0 cm\hat{ }2, 4.5 cm\hat{ }2, or 5.0 cm\hat{ }2. For example, the individual is selected for having a wound area of at least 0.5 cm\hat{ }2. In some cases, the individual is selected for having a wound area of not more than 1.0 cm\hat{ }2, 1.5 cm\hat{ }2, 2.0 cm\hat{ }2, 2.5 cm\hat{ }2, 3.0 cm\hat{ }2, 3.5 cm\hat{ }2, 4.0 cm\hat{ }2, 4.5 cm\hat{ }2, or 5.0 cm\hat{ }2. For example, the individual is selected for having a wound area of not more than 4.0 cm\hat{ }2. Often, the individual is selected for having a wound having a Texas wound stage comprising 1a, 1c, or 2a. In some cases, the individual is selected for having a wound on the leg, ankle, or foot. In some cases, the individual is selected for having a wound located distal to the malleolus. In some cases, the individual is selected for having a pressure in a toe of an affected limb is at least 20 mmHg, 30 mmHg, 40 mmHg, 50 mmHg, or 60 mmHg. For example, the individual is selected for having a pressure in a toe of an affected limb is at least 40 mmHg. Often, the individual is selected for having an ankle-brachial systolic pressure index is from about 0.5 to about 1.5, about 0.5 to about 1.4, about 0.5 to about 1.3, about 0.5 to about 1.2, about 0.5 to about 1.1, about 0.6 to about 1.5, about 0.6 to about 1.4, about 0.6 to about 1.3, about 0.6 to about 1.2, about 0.6 to about 1.1, about 0.7 to about 1.5, about 0.7 to about 1.4, about 0.7 to about 1.3, about 0.7 to about 1.2, about 0.7 to about 1.1, about 0.8 to about 1.5, about 0.8 to about 1.4, about 0.8 to about 1.3, about 0.8 to about 1.2, about 0.8 to about 1.1, about 0.9 to about 1.5, about 0.9 to about 1.4, about 0.9 to about 1.3, about 0.9 to about 1.2, or about 0.9 to about 1.1. For example, the individual is selected for having an ankle-brachial systolic pressure index is from about 0.7 to about 1.3. Sometimes, the individual is selected for having a diagnosis of peripheral neuropathy, for example a diagnosis of peripheral neuropathy using ADA guidelines. Often, the wound is fully closed after a single administration. In some cases, the wound is fully closed after a second administration of the composition. Often, the wound is an open wound, a non-self-healing wound, a dermal wound, an ulcerative wound, a diabetic wound, a venous ulcer, and a pressure ulcer.

Further provided herein are methods of treating a wound in an individual comprising administering a stromal stem cell composition provided herein, wherein the individual is selected when they are observed to have a life expectancy of greater than 12 months. Often an individual is selected for treatment when they have no diagnosis of an immunodeficiency disorder. In some cases, an individual is selected for treatment when they are observed to have a negative hepatitis B surface antigen and hepatitis C antibody test results. In some cases, an individual is selected for treatment when they are observed to have no symptoms of a connective tissue disease. Often, an individual is selected for treatment when they are observed to have a serum creatinine of less than 220 μmol/L, for example less than 100 μmol/L, less than 150 μmol/L, less than 200 μmol/L, or less than 220 μmol/L. Often individuals are selected for treatment when they are observed to have AST of less than 10 times the upper limit of normal. In some cases, individuals are selected for treatment when they are observed to have ALT of less than 10 times the upper limit of normal. Sometimes, individuals are selected for treatment when they are observed to have ALP of less than 10 times the upper limit of normal. Often individuals are selected for treatment when they are observed to have a serum albumin of greater than 30 mg/dL. In some cases, individuals are selected for treatment when they are observed to have a serum albumin of greater than 20 mg/dL, 25 mg/dL, 30 mg/dL, 35 mg/dL, or more. In some cases, individuals are selected for treatment when they are observed to have no history of cancer in the past five years. In some cases, individuals are selected for treatment when they are observed to have no active wound infection, for example no recent onset of erythema, edema, or increased temperature. In some cases, individuals are selected for treatment when they are observed to have no diagnosis of Diabetic Charcot neuroarthropathy or other structural deformity preventing adequate off-loading. In some cases, individuals are selected for treatment when they are observed to have no treatment with systemic corticosteroid immunosuppressive agent, antiviral agent, or radiation therapy. In some cases, individuals are selected for treatment when they are observed to have no wounds caused primarily by untreated vascular insufficiency. Often, individuals are selected for treatment when they are observed to have wounds with an etiology not related to diabetes. In some cases, individuals are selected for treatment when they are observed to have fewer than three wounds on the lower extremity. Often, individuals are selected for treatment when they are observed to have ulcers with underlying osteomyelitis on the leg with the wound treated. In some cases, individuals are selected for treatment when they are observed to have cellulitis, suppurative inflammation, erythema, tenderness, malaise, chills, or fever. Often, individuals are selected for treatment when they are observed to have no revascularization surgery on the leg with the wound. In some cases, individuals are selected for treatment when they are observed to have no history of surgery to lengthen Achilles tendon on the leg with the wound treated. Often, individuals are selected for treatment when they are observed to have no signs of necrosis, purulence, or sinus tracts which cannot be removed by debridement on foot treated. In some cases, individuals are selected for treatment when they are observed to have not received dermal substitute or living skin equivalent, for example Leukopatch. In some cases, individuals are selected for treatment when they are observed to have no history of PDGF-BB therapy.

In methods of treatment of wounds using stromal stem cells provided herein, individuals treated with stromal stem cells are observed in the time to complete ulcer closure. In some cases, individuals treated with stromal stem cells are observed in ulcer healing trajectory over time. Often, individuals treated with stromal stem cells are observed in absolute and percent changes in ulcer area. In some cases, individuals treated with stromal stem cells are observed in the durability of ulcer closure. Often individuals treated with stromal stem cells are observed every week, every 2 weeks after treatment, every 3 weeks after treatment, every 4 weeks after treatment, every 5 weeks after treatment, or every 6 weeks after treatment.

In methods of treatment of wounds provided herein, if it is medically necessary the ulcer is debrided prior to ulcer assessment. In some cases, the method comprises converting the ulcer environment from that of a chronic wound to that of an acute wound. Often, the method comprises treating the ulcer site as soon as possible after debridement/scoring of the wound bed to take advantage of the influx of blood into the ulcer. Often, the method comprises sharp surgical debridement to remove all necrotic soft tissue, byperkeratotic wound margins, bacterial burden, cellular debris, sinus tracts, fistulae, undermined borders, and callus to produce viable wound margins and a clean ulcer site. In some cases, debridement is not necessary and the method comprises lightly scoring the ulcer site and margins to create the small influx of blood that is needed to facilitate ulcer healing. In some cases, mechanical, enzymatic, biological, or autolytic debridement. Often the method comprises applying pressure to stop bleeding. In many cases, silver nitrate is not used to stop ulcer site bleeding.

In methods of treatment of wounds provided herein, the method often comprises measuring ulcer area the acetate tracing method. In some cases, the method comprises taking a photograph of the wound. Often, the method comprises using a Silhouette device. In many cases, the method comprises using sterile saline to remove any debris left after debridement. In some cases, the method comprises irrigating the ulcer with saline and wiping debris from the ulcer with sterile gauze. In some cases, the method comprises using care to minimize mechanical trauma at the site. Often, the method comprises using moist (sterile saline) sterile gauze to keep the ulcer moist until the treatment is administered. In some cases, the method comprises observing blood in the ulcer to facilitate the effects of the treatment. Often, the method comprises using a dressing that will maintain a moist wound-healing environment, manage wound exudates, and protect the peri-ulcer skin (e.g. Opsite/Mefix). In some cases, the method comprises cutting sterile gauze in the approximate size and shape of the ulcer, and placing the sterile gauze over the ulcer. In some cases, the method comprises wrapping the dressing with a non-adberent bandage, such as Mepilex® neutral foam, to secure the wound dressing. In some cases, the method comprises placing a white sock on the foot so exudate can be visualized should it occur.

In some cases, the method comprises cleansing the ulcer with saline and a moist sterile gauze pad each time the dressing is changed. In some cases, the method comprises performing culture and sensitivity tests. In some cases, the methods comprise administration of an appropriate oral and/or intravenous antimicrobial (other than antivirals). Often, the method comprises systemic administration of antimicrobial therapy for suspected or documented infections. In some cases, the method comprises directing the individual to wear a special weight off-loading orthopedic shoe (DH Aircast Walker) until at least 2 weeks after ulcer closure. In some cases, the method comprises directing the individual to wear a special weight off-loading orthopedic shoe for at least 2 weeks, at least 4 weeks, at least 6 weeks, at least 8 weeks, at least 10 weeks, at least 12 weeks, or longer.

Also provided herein, are methods of treating a wound in an individual comprising administering stromal stem cell compositions herein wherein the composition comprises 1% to 10% collagen. For example, in some cases, the method comprises administering the stromal stem cells in a composition comprising 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, or 10% collagen. In some cases, the method comprises administering the stromal stem cells in a composition comprising 2.6% collagen.

In some cases, methods of treating wounds herein comprise administering compositions for treating wounds comprise stromal stem cells, wherein the population of stromal stem cells in the composition is at least 30% SDC2+. In some cases, the method comprises administering a composition wherein the population of stromal stem cells in the composition is at least 40% SDC2+. Sometimes, the method comprises administering a composition wherein the population of stromal stem cells in the composition is at least 50% SDC2+. Often, the method comprises administering a composition wherein the population of stromal stem cells in the composition is at least 60% SDC2+. Sometimes, the method comprises administering a composition wherein the population of stromal stem cells in the composition is at least 70% SDC2+. In some cases, the method comprises administering a composition wherein the population of stromal stem cells in the composition is at least 80% SDC2+. In some cases, the method comprises administering a composition wherein the population of stromal stem cells in the composition is at least 90% SDC2+. Sometimes, the method comprises administering a composition wherein the population of stromal stem cells in the composition is at least 95% SDC2+. In some cases, the method comprises administering a composition wherein the population of stromal stem cells in the composition is at least 97% SDC2+. Often, the method comprises administering a composition wherein the population of stromal stem cells in the composition is at least 99% SDC2+. In some cases, the method comprises administering a composition wherein the population of stromal stem cells in the composition is substantially 100% SDC2+.

In some cases, methods of treating wounds herein comprise administering compositions for treating wounds are provided in therapeutically effective doses. Often, the method comprises administering a composition wherein the composition comprises at least 10\hat{ }3 stromal stem cells. Sometimes, the method comprises administering a composition wherein the composition comprises at least 10\hat{ }4 stromal stem cells. In some cases, the method comprises administering a composition wherein the composition comprises at least 1045 stromal stem cells. Often, the method comprises administering a composition wherein the composition comprises at least 10\hat{ }6 stromal stem cells. Sometimes, the method comprises administering a composition wherein the composition comprises at least 10\hat{ }7 stromal stem cells. In some cases, the method comprises administering a composition wherein the composition comprises at least 10.6×10\hat{ }6 stromal stem cells per 3 cm\hat{ }2 wound. In some cases, the method comprises administering a composition wherein the composition comprises at least 1×10\hat{ }6 stromal stem cells per 3 cm\hat{ }2, 2×10\hat{ }6 stromal stem cells per 3 cm\hat{ }2, 3×10\hat{ }6 stromal stem cells per 3 cm\hat{ }2, 4×10\hat{ }6 stromal stem cells per 3 cm\hat{ }2, 5×10\hat{ }6 stromal stem cells per 3 cm\hat{ }2, 6×10\hat{ }6 stromal stem cells per 3 cm\hat{ }2, 7×10\hat{ }6 stromal stem cells per 3 cm\hat{ }2, 8×10\hat{ }6 stromal stem cells per 3 cm\hat{ }2, 9×10\hat{ }6 stromal stem cells per 3 cm\hat{ }2, 10,×10\hat{ }6 stromal stem cells per 3 cm\hat{ }2, 10.6×10\hat{ }6 stromal stem cells per 3 cm\hat{ }2, 11×10\hat{ }6 stromal stem cells per 3 cm\hat{ }2, or 12×10\hat{ }6 stromal stem cells per 3 cm\hat{ }2 wound. In some cases, the method comprises administering a composition wherein the composition comprises at least 10.6×10\hat{ }6 stromal stem cells per 3 cm\hat{ }2 wound. In some cases, the method comprises administering the cells as a continuous film over the entire ulcer surface area, to the margins.

Compositions for treating wounds further comprise a pharmaceutically acceptable excipient, in some cases, to prepare a salve or an ointment. Sometimes, the composition comprises an extracellular matrix component. Often, the extracellular matrix component is a hyaluronic acid. Sometimes, the extracellular matrix component is a collagen. In some cases, the collagen is a collagen matrix. In some cases, the stromal stem cells are suspended in the collagen matrix. Often, the composition is a salve or an ointment. Often, the composition is affixed to a backing.

Methods of treatment herein comprise one or more administrations of stromal stem cell compositions in doses disclosed herein. In some cases, methods comprise one administration of stromal stem cell compositions. In some cases, methods comprise two administrations of stromal stem cell compositions. Often, methods comprise three administrations of stromal stem cell compositions. Sometimes, methods comprise four administrations of stromal stem cell compositions. Often, methods comprise five administrations of stromal stem cell compositions. In some cases, methods comprise six administrations of stromal stem cell compositions. In some cases, one or more administrations of stromal stem cells are administered daily. Sometimes, one or more administrations of stromal stem cells are administered weekly. Sometimes, one or more administrations of stromal stem cells are administered biweekly. In some cases, one or more administrations of stromal stem cells are administered monthly. In some cases, one or more administrations of stromal stem cells are administered every three months. Often, one or more administrations of stromal stem cells are administered every six months. Sometimes, one or more administrations of stromal stem cells are administered yearly. In many cases, the wound is fully closed after a single administration of the composition. Often, the wound if fully closed after a second administration of the composition. In some cases, the wound if fully closed after a third administration of the composition.

Stromal stem cell treatments for wounds are administered via a route chosen by a physician or one of skill in the art. In some cases, the compositions are administered topically. In some cases, the compositions are injected subcutaneously, intradermally, or intramuscularly at the site of the wound. In some cases, the method comprises applying a dressing to the composition and the wound.

Stromal stem cell compositions for parenteral administration herein comprise pharmaceutically acceptable excipients suitable for such routes of administration. Such excipients, stabilize, preserve, and protect the stromal stem cells in the composition. In some cases, compositions comprise glucose, a buffer, sodium chloride, dimethylsulfoxide, and/or glycerol.

### Stromal Stem Cell Compositions for Topical Administration

Also provided herein are stromal stem cell compositions comprising at least one stromal stem cell and an extracellular matrix. In some cases, stromal stem cell compositions herein comprise a backing. In some cases, the extracellular matrix comprises collagen or hyaluronic acid. Often, the composition comprises at least 1%, 2%, 3%, 4%, 5%, 6%, 7%, 8%, 9%, 10%, or more collagen. Sometimes, the composition comprises 1-2%, 1-3%, 1-4%, 1-5%, 1-6%, 1-7%, 1-8%, 1-9%, 1-10%, 2-3%, 2-4%, 2-5%, 2-6%, 2-7%, 2-8%, 2-9%, 2-10%, 3-4%, 3-5%, 3-6%, 3-7%, 3-8%, 3-9%, 3-10%, 4-5%, 4-6%, 4-7%, 4-8%, 4-9%, 4-10%, 5-6%, 5-7%, 5-8%, 5-9%, 5-10%, 6-7%, 6-8%, 6-9%, 6-10%, 7-8%, 7-9%, 7-10%, 8-9%, 8-10%, or 9-10% collagen. Often, the composition comprises at least 2% collagen. Sometimes, the backing comprises an adhesive. In some cases, the stromal stem cells are suspended in the extracellular matrix.

Stromal stem cell compositions herein comprise stromal stem cells that at least 30% SDC2+. In some cases, the stromal stem cells are at least 40% SDC2+. Often, the stromal stem cells are at least 50% SDC2+. Sometimes, the stromal stem cells are at least 60% SDC2+. Often, the stromal stem cells are at least 70% SDC2+. In some embodiments, the stromal stem cells are at least 80% SDC2+. In some cases, the stromal stem cells are at least 90% SDC2+. Often, the stromal stem cells are at least 95% SDC2+. Sometimes, the stromal stem cells are at least 99% SDC2+. In some cases. the stromal stem cells are 100% SDC2+.

Compositions herein are provided in therapeutically effective doses. Sometimes, compositions comprise at least 10\hat{ }3 stromal stem cells. Often, the composition comprises at least 10\hat{ }4 stromal stem cells. Sometimes, the composition comprises at least 10\hat{ }5 stromal stem cells. In some cases, the composition comprises at least 10\hat{ }6 stromal stem cells. Often, the composition comprises at least 10\hat{ }7 stromal stem cells In some cases, the composition comprises 10\hat{ }3 to 10\hat{ }4, 10\hat{ }3 to 10\hat{ }5, 10\hat{ }3 to 10\hat{ }6, 10\hat{ }3 to 10\hat{ }7, 10\hat{ }4 to 10\hat{ }5, 10\hat{ }4 to 10\hat{ }6, 10\hat{ }4 to 10\hat{ }7, 10\hat{ }5 to 10\hat{ }6, 10\hat{ }5 to 10\hat{ }7, or 10\hat{ }6 to 10\hat{ }7 stromal stem cells.

Also provided herein are methods of preparing wound dressings for a wound comprising: (a) obtaining a first composition comprising at least 10\hat{ }3 stromal stem cells; (b) obtaining a second composition comprising at least 6% collagen; (c); preparing a mixture of the first composition and the second composition; and (d) applying the resulting mixture to a backing having an adhesive for applying to the wound. Sometimes, the dressing is for treatment of a diabetic wound. Often, the resulting mixture comprises 10\hat{ }3 to 10\hat{ }4, 10\hat{ }3 to 10\hat{ }5, 10\hat{ }3 to 10\hat{ }6, 10\hat{ }3 to 10\hat{ }7, 10\hat{ }4 to 10\hat{ }5, 10\hat{ }4 to 10\hat{ }6, 10\hat{ }4 to 10\hat{ }7, 10\hat{ }5 to 10\hat{ }6, 10\hat{ }5 to 10\hat{ }7, or 10\hat{ }6 to 10\hat{ }7 stromal stem cells. Sometimes, the resulting mixture comprises at least 10\hat{ }3 stromal stem cells. Often, the resulting mixture comprises 1-2%, 1-3%, 1-4%, 1-5%, 1-6%, 1-7%, 1-8%, 1-9%, 1-10%, 2-3%, 2-4%, 2-5%, 2-6%, 2-7%, 2-8%, 2-9%, 2-10%, 3-4%, 3-5%, 3-6%, 3-7%, 3-8%, 3-9%, 3-10%, 4-5%, 4-6%, 4-7%, 4-8%, 4-9%, 4-10%, 5-6%, 5-7%, 5-8%, 5-9%, 5-10%, 6-7%, 6-8%, 6-9%, 6-10%, 7-8%, 7-9%, 7-10%, 8-9%, 8-10%, or 9-10% collagen. In some cases, the resulting mixture comprises 2.6% collagen. In some cases, the resulting mixture comprises a collagen matrix. In some cases, the mixing comprises stirring. In some cases, the mixing comprises shaking. In some cases, the mixing comprises inverting the mixture of components. In some embodiments, the mixing comprises passing each component between two luer lok syringes.

Stromal stem cell compositions herein comprise stromal stem cells that at least 30% SDC2+. Sometimes, the stromal stem cells are at least 40% SDC2+. Often, the stromal stem cells are at least 50% SDC2+. In some cases, the stromal stem cells are at least 60% SDC2+. Often, the stromal stem cells are at least 70% SDC2+. Sometimes, the stromal stem cells are at least 80% SDC2+, Often, the stromal stem cells are at least 90% SDC2+. In some cases, the stromal stem cells are at least 95% SDC2+. Sometimes, the stromal stem cells are at least 99% SDC2+. Often, the stromal stem cells are 100% SDC2+.

### Preparation of Stromal Stem Cells for Therapeutic Use

Stromal stem cells for therapeutic are isolated from human umbilical cord tissue (UCT) by selecting for cells expressing CD362 (SDC2). These cells often have a higher proliferative capacity compared to bone marrow derived mesenchymal stem cells. This represents a significant increase in the potential cell yield available for therapeutic dosing from each cord used.

Methods of preparing stromal stem cells for therapeutic use herein, in some cases comprise obtaining an umbilical cord tissue sample. Often, the method comprises sterilizing the umbilical cord sample. In some cases, the method comprises dividing the umbilical cord sample into a known weight. Often, the method comprises further cutting each portion of umbilical cord into small pieces. In some cases, the method comprises cutting each portion of umbilical cord into pieces no larger than 0.1 mm\hat{ }2, 0.2 mm\hat{ }2, 0.3 mm\hat{ }2, 0.4 mm\hat{ }2, 0.5 mm\hat{ }2, 0.6 mm\hat{ }2, 0.7 mm\hat{ }2, 0.8 mm\hat{ }2, 0.9 mm\hat{ }2, or 1.0 mm\hat{ }2. In some cases, the method comprises cutting each portion of umbilical cord into pieces about 0.5 to about 1 mm\hat{ }2. In some cases, the method comprises removing any blood from the umbilical cord samples. In some cases, the method comprises mixing the pieces of umbilical cord with a protease, such as collagenase, trypsin, proteinase K, or other protease. Often, the umbilical cord samples are incubated with a protease for at least 30, 35, 40, 45, 50, 55, or 60 minutes. In some cases, the method comprises stopping the protease reaction with a cell culture media comprising serum. Often the method comprises filtering the protease treated umbilical cord samples through a cell strainer, such as a 100 μm cell strainer, resulting in a solution comprising the cells from the umbilical cord.

Method of preparing stromal stem cells for therapeutic use herein comprise labeling the dissociated umbilical cord cells with an agent that binds to SDC2, such as an anti-SDC2 antibody. In some cases, cells labeled with the anti-SDC2 cell antibody are separated from the unlabeled umbilical cord cells. In some cases, labeled cells are separated from unlabeled cells using fluorescence activated cell sorting. In some cases, labeled cells are separated from unlabeled cells using a magnetic cell separating device. In some cases, labeled cells are separated from unlabeled cells using a MACSQuant Tyto device. Often, dead cells are removed from the cells during the cell separation step. In some cases, the cell separation step comprises an enrichment sort. In some cases, the cell separation step comprises an enrichment sort and a purity sort. In some cases, the sorted cells are counted. Often, the method comprises culturing the sorted cells to expand cell numbers.

Stromal stem cells and methods and uses in treating disease are illustrated by the drawings provided herein. FIG. 1 provides a graph depicting the number of individuals needing a liver transplant each year due to illness with primary biliary cholangitis (PBC), primary sclerosing cholangitis (PSC), and autoimmune hepatitis (AIH). On the x-axis is year from 1994 to 2014 and on the y-axis is normalized transplant listings per year from 0 to 150. The number of liver transplants needed due to PBC has decreased, while the number of liver transplants needed due to PSC and AIH has increased over the reported time period. This figure indicates that there is increased need for liver transplants in patients observed to have PSC and AIH.

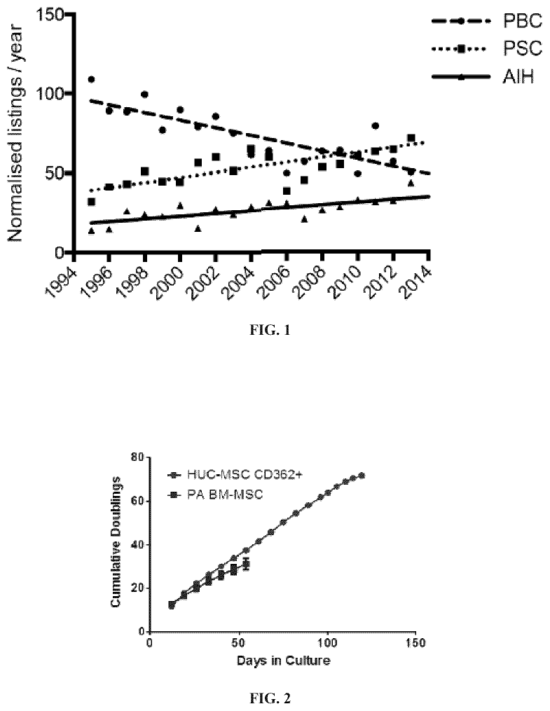

FIG. 2 shows the comparison in cumulative doublings for CD362+ (SDC2+) stromal stem cells isolated from umbilical cord blood (HUC-MSC CD362+) compared to bone marrow derived stromal stem cells (PA BM-MSC). The CD362+ umbilical cord blood derived stromal stem cells show increased doublings compared to the bone marrow derived stromal stem cells. This figure indicates that CD362+ stromal stem cells from umbilical cord provide advantages compared to CD362+ stromal stem cells isolated from bone marrow.

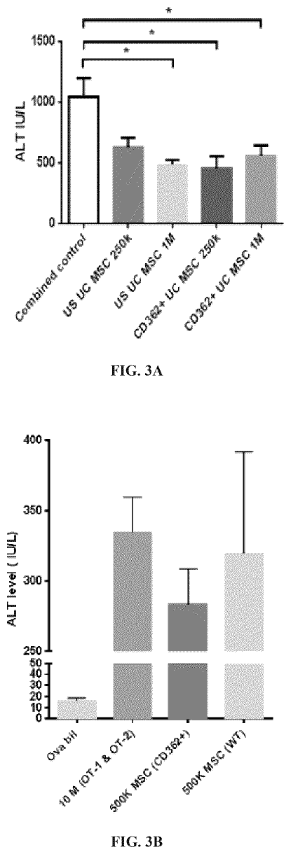

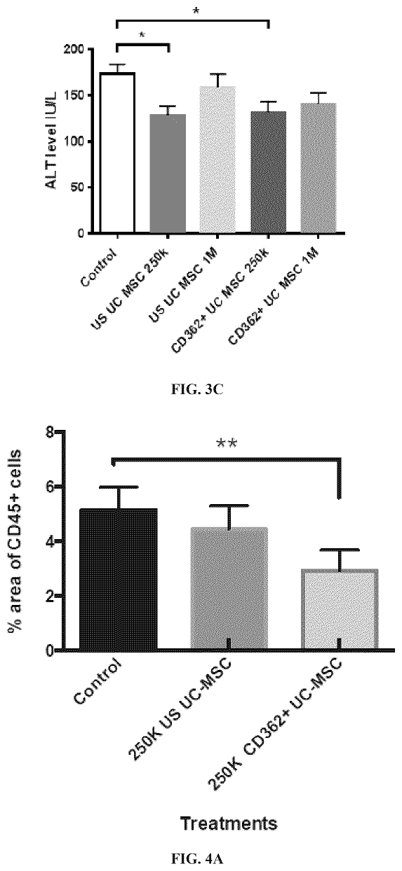

FIG. 3A shows a graph depicting reduced serum ALT in MDR2KO/FVB mice treated with CD362+ (SDC2+) mesenchymal stem cells compared to control. One control bar is shown and four treatment bars are shown. The treatments are US UC MSC 250,000; US UC MSC 1,000,000; CD362+ UC MSC 250,000; and CD362+ UCMSC 1,000,000. The treatment with 1,000,000 US UC MSC is comparable to the treatments with 250,000 CD362+ UC MSC and 1,000,000 CD362+ UC MSC; and all three are significantly different from the control (marked with a *). This figure indicates that MDR2KO/FVB mice treated with CD362+stromal stem cells are observed to have improvement compared to MDR2KO/FVB mice treated with a control.

FIG. 3B shows a graph depicting reduced serum ALT in Ova-bil mice treated with CD362+ (SDC2+) mesenchymal stem cells. One bar is shown for mice without liver disease. The second bar shows diseased mice given no stromal stem cell treatment have increased ALT levels. The third bar shows diseased mice given 500,000 CD362+ stromal stem cells having reduced ALT levels. The fourth bar shows diseased mice given unsorted stromal stem cells having slightly reduced ALT levels. This figure indicates that Ova-bil mice treated with CD362+ stromal stem cells are observed to have improved liver function.

FIG. 3C shows a graph depicting reduced serum ALT in acute CCL4 mice treated with CD362+ (SDC2+) mesenchymal stem cells. The first bar shows untreated mice having elevated ALT levels. The second bar shows mice treated with 250,000 unsorted stromal stem cells having reduced ALT levels. The third bar shows mice treated with 1,000,000 unsorted stromal stem cells having slightly reduced ALT levels. The fourth and fifth bars shown mice treated with 250,000 or 1,000,000 CD362+ (SDC2+) stromal stem cells having reduced ALT levels. Significant changes are marked with an *. This figure indicates that CCL4 mice treated with CD362+ stromal stem cells have improved liver function.

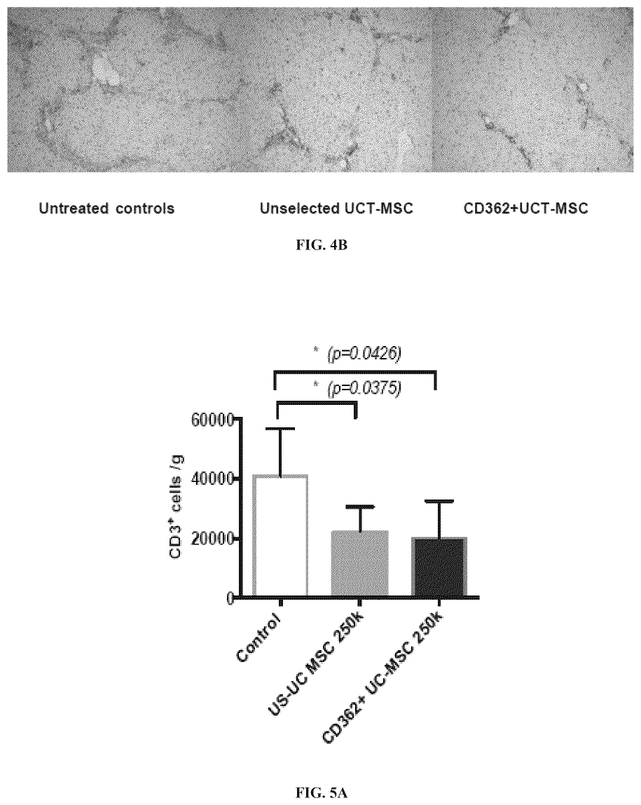

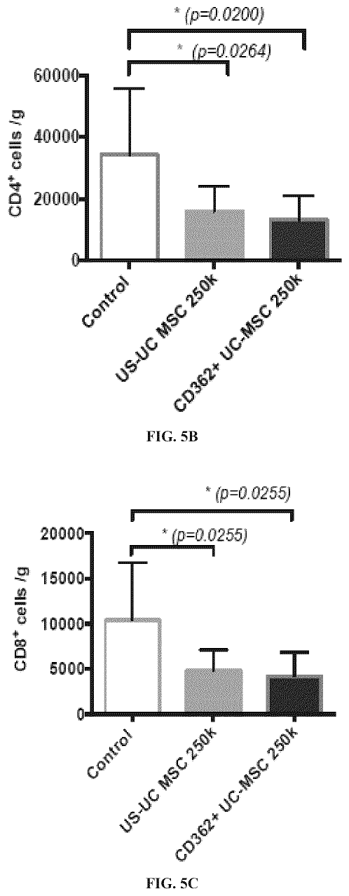

FIG. 4A shows a graph depicting reduction in histological inflammation in MDR2KO/FVB mice treated with CD362+ (SDC2+) mesenchymal stem cells. The first bar is data for untreated mice. The second bar is data for mice treated with 250,000 unsorted stromal stem cells. The third bar is data for mice treated with 250,000 CD362+ (SDC2+) stromal stem cells showing a significant reduction in CD45+ cells. This figure indicates that MDR2KO/FVB mice treated with CD362+ have improved liver histology.

FIG. 4B shows a series of photomicrographs depicting reduction in histological liver injury in MDR2KO/FVB mice treated with CD362+ (SDC2+) mesenchymal stem cells. The first panel shows a photomicrograph from an untreated mouse liver showing darkened cells where inflammatory cells are present. The center panel shows a photomicrograph from a mouse treated with unselected stromal stem cells showing a slight reduction in inflammatory cells. The right panels show a photomicrograph from a mouse treated with CD362+ (SDC2+) stromal stem cells having reduced inflammatory cells. This figure indicates that MDR2KO/FVB mice treated with CD362+ stromal stem cells have reduced inflammation in the liver.

FIG. 5A shows a graph depicting a reduction in hepatic CD3+ T cells in mice treated with CD362+ (SDC2+) mesenchymal stem cells. The first bar shows untreated mice having elevated CD3+ T cells. The center bar shows mice treated with unsorted stromal stem cells having reduced CD3+ T cells. The last bar shows mice treated with CD362+ (SDC2+) having reduced CD3+ T cells. This figure indicates that mice treated with CD362+ stromal stem cells have reduced inflammation in the liver.

FIG. 5B shows a graph depicting a reduction in hepatic CD4+ T cells in mice treated with CD362+ (SDC2+) mesenchymal stem cells. The first bar shows untreated mice having elevated CD4+ T cells. The center bar shows mice treated with unsorted stromal stem cells having reduced CD4+ T cells. The last bar shows mice treated with CD362+ (SDC2+) having reduced CD4+ T cells. This figure indicates that mice treated with CD362+ stromal stem cells have reduced inflammation in the liver.

FIG. 5C shows a graph depicting a reduction in hepatic CD8+ T cells in mice treated with CD362+ (SDC2+) mesenchymal stem cells. The first bar shows untreated mice having elevated CD8+ T cells. The center bar shows mice treated with unsorted stromal stem cells having reduced CD8+ T cells. The last bar shows mice treated with CD362+ (SDC2+) having reduced CD8+ T cells. This figure indicates that mice treated with CD362+ stromal stem cells have reduced inflammation in the liver.

FIG. 6 shows a graph depicting an increase in regulatory T cells in mice treated with CD362+ (SDC2+) mesenchymal stem cells. The first data point is control mice showing about 5% CD3+ CD4+ CD25^{high} FOXP3+ cells. The second data point shows mice treated with unsorted stromal stem cells having increased CD3+ CD4+ CD25^{high} FOXP3+ cells (about 12%). The last data point shows mice treated with CD362+ stromal stem cells having increased CD3+ CD4+ CD25^{high} FOXP3+ cells (about 13%). This figure indicates that mice treated with CD362+ stromal stem cells have reduced inflammation in the liver.

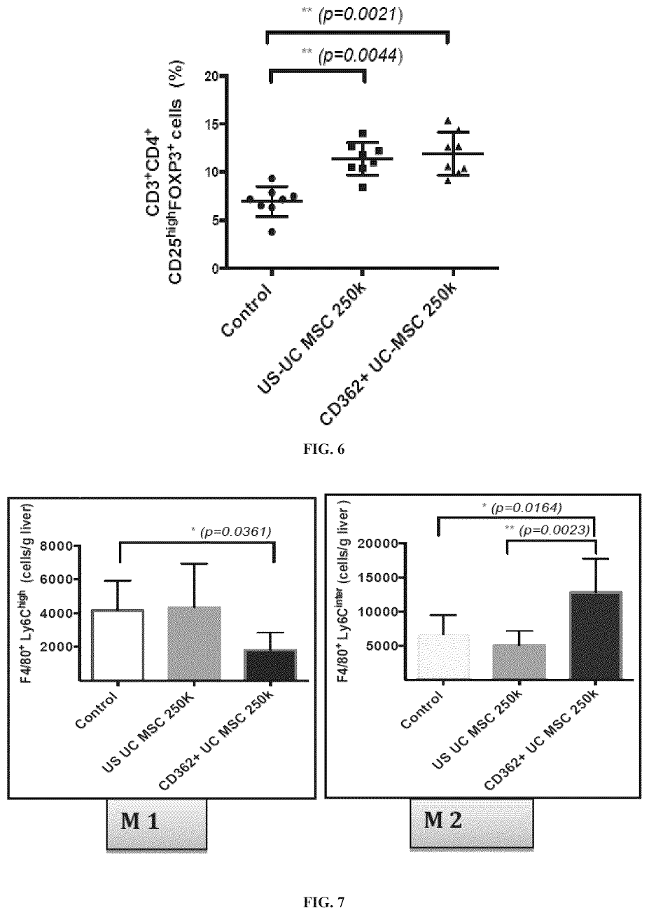

FIG. 7 shows a series of graphs depicting a reduction in liver macrophage M2 polarization in mice treated with CD362+ (SDC2+) mesenchymal stem cells. The first panel shows a decrease in M1 macrophages in treated mice as measured by F4/80+ Ly6C^{high} cells/g liver. The first bar is data for untreated mice showing increased M1 cells. The second bar is data for mice treated with unsorted stromal stem cells showing no reduction in M1 cells. The third bar is data for mice treated with CD362+ stromal stem cells showing reduced M1 cells (significant to p=0.0361). The second panel shows an increase in M2 macrophages in treated mice as measured by F4/80+ Ly6C^{inter} cells/g liver. The first bar is data for untreated mice showing reduced M2 cells. The second bar is data for mice treated with unsorted stromal stem cells showing no increase in M2 cells. The third bar is data for mice treated with CD362+ stromal stem cells showing increased M2 cells (significant to p=0.0023). This figure indicates that mice treated with CD362+ stromal stem cells have reduced inflammation in the liver.

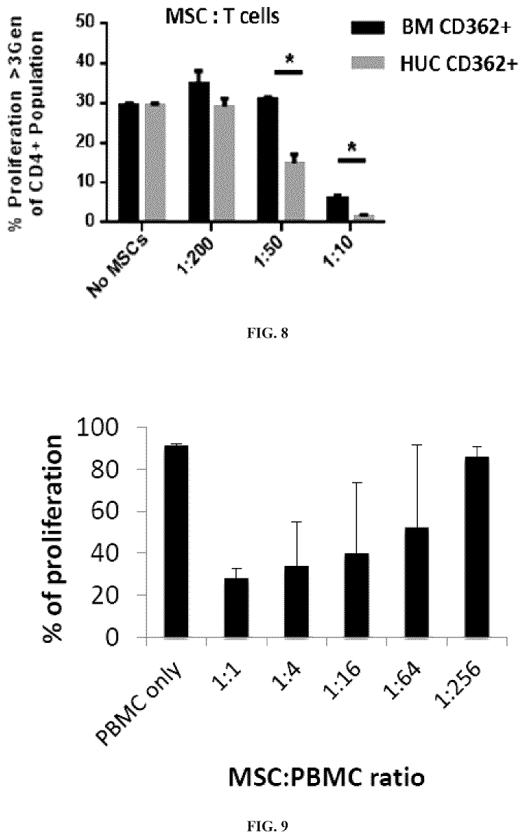

FIG. 8 shows a graph depicting reduction in T cell proliferation in healthy volunteer CD4+ T cells treated with CD362+ (SDC2+) mensenchymal stem cells. The graph shows T cells treated with CD362+ mesenchymal stem cells in ratios of 1:200, 1:50 and 1:10, and no mesenchymal stem cells. Black bars depict cells treated with bone marrow mesenchymal stem cells. Grey bars depict cells treated with umbilical cord blood mesenchymal stem cells. A reduction in proliferation is observed in cells treated with increasing amounts of mesenchymal stem cells. Umbilical cord mesenchymal stem cells show a greater reduction in proliferation than bone marrow mesenchymal stem cells. This figure indicates that cells treated with CD362+ stromal stem cells from umbilical cord are more effective in reducing inflammation.

FIG. 9 shows a graph depicting a reduction in T cell proliferation in PSC volunteer PBMCs treated with CD362+ (SDC2+) mensenchymal stem cells. The graph shows PBMCs treated with CD362+ mesenchymal stem cells in ratios of 1:1, 1:4, 1:16, 1:64, and 1:256, and no PBMCs only. Black bars depict cells treated with bone marrow mesenchymal stem cells. Grey bars depict cells treated with umbilical cord blood mesenchymal stem cells. A reduction in proliferation is observed in cells treated with increasing amounts of mesenchymal stem cells. Umbilical cord mesenchymal stem cells show a greater reduction in proliferation than bone marrow mesenchymal stem cells. This figure indicates that cells treated with CD362+ stromal stem cells from umbilical cord are more effective in reducing inflammation.

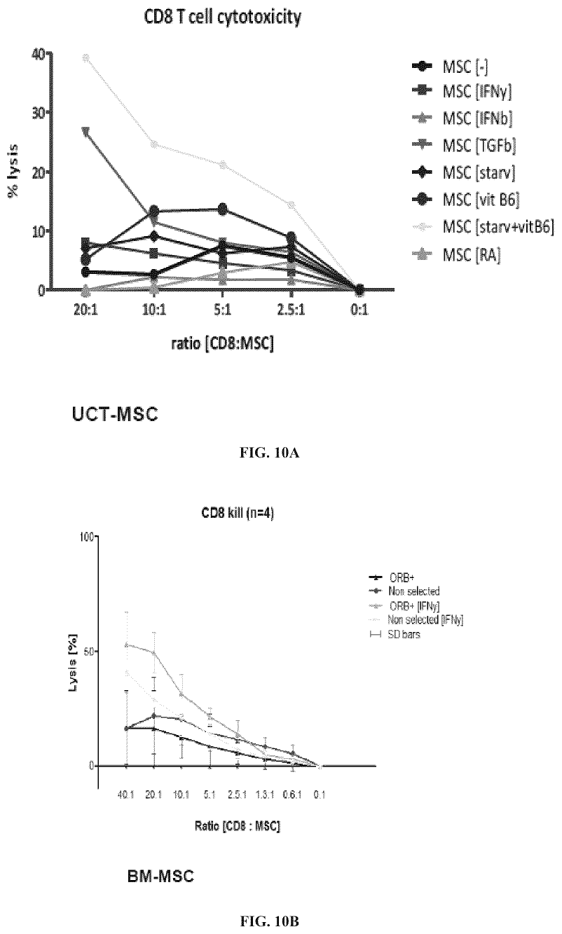

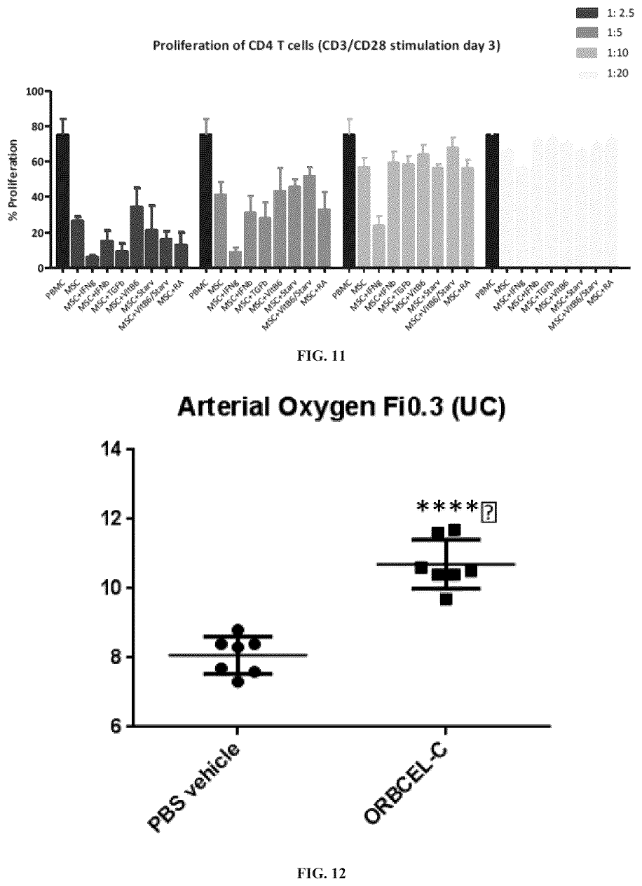

FIG. 10A shows a graph depicting reduction in CD8+ T cell lysis in CD8+ T cells incubated with CD362+ (SDC2+) mensenchymal stem cells. The graph shows CD362+ mesenchymal stem cells have reduced immunogenicity as measured by lysis in a lysis assay using ratios of 20:1, 10:1, 5:1, 2.5:1 and 0:1. Cells treated with starvation and vitamin B6 were most immunogenic. Cells treated with TGFb were next most immunogenic. Cells treated with no treatment, IFNγ, IFNb, starvation, vitamin B6, or retinoic acid had relatively low immunogenicity with IFNb and retinoic acid having the lowest values. This figure indicates that CD362+ stromal stem cells have low immunogenicity.

FIG. 10B shows a graph depicting reduction in CD8+ T cell lysis in CD8+ T cells incubated with CD362+ (SDC2+) mensenchymal stem cells. The graph shows CD362+ mesenchymal stem cells have reduced immunogenicity as measured by lysis in a lysis assay using ratios of 40:1, 20:1, 10:1, 5:1, 2.5:1, 1.3:1, 0.6:1, and 0:1. ORB+ cells treated with IFNγ had the highest immunogenicity. Non-selected cells treated with IFNγ had the next lowest immunogenicity. Non-selected cells that were not treated had the next lowest immunogenicity. The lowest immunogenicity was observed with untreated ORB+ cells. This figure indicates that CD362+ stromal stem cells have low immunogenicity.

FIG. 11 shows a graph depicting a reduction in CD4+ T cell proliferation in cells incubated with CD362+ (SDC2+) mensenchymal stem cells treated with various pretreatment regimens including IFNγ, IFNb, TGFb, vitamin B6, starvation, vitamin B6 and starvation, and retinoic acid. CD4+ T cells were incubated with CD362+ mesenchymal stem cells in ratios of 1:2.5 (first group), 1:5 (second group), 1:10 (third group), 1:20 (fourth group), and PBMCs alone (first bar of each group). At almost every ratio, mesenchymal stem cells incubated with IFNγ showed the least CD4 T cell proliferation. This figure indicates that CD362+ stromal stem cells have low immunogenicity.

FIG. 12 shows a graph depicting an increase in arterial oxygen FI0.3 (UC) in Sprague Dawley rats with lung injury treated with CD362+ (SDC2+) mensenchymal stem cells. The graph shows arterial oxygen on the Y axis and compares mice having a lung injury treated with PBS vehicle to mice treated with CD362+ mesenchymal stem cells. This figure indicates that CD362+ stromal stem cells are effective in treating lung injury.

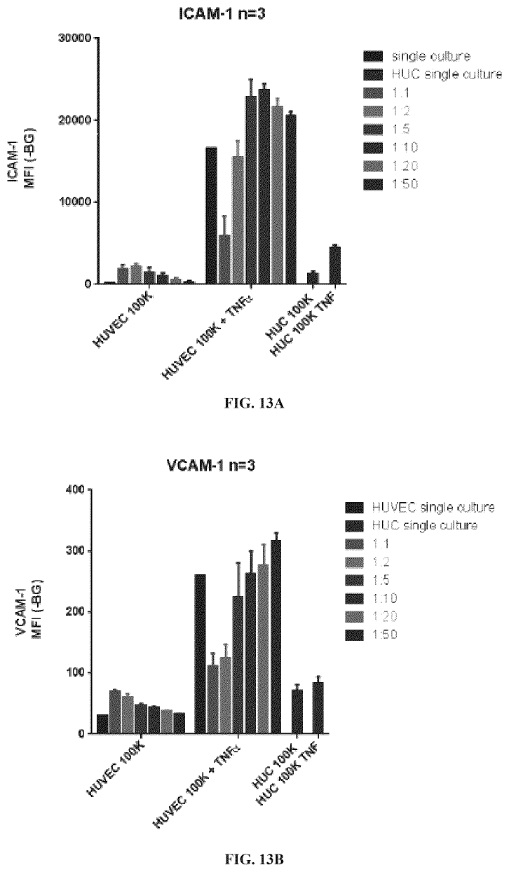

FIG. 13A shows a graph depicting a decrease in TNF-alpha induced ICAM-1 in endothelial cells incubated with mesenchymal stem cells. The graph shows incubation in ratios of 1:1, 1:2, 1:5, 1:10, 1:20, and 1:50, as well as single culture of mesenchymal stem cells and endothelial cells. The bars from left to right in this graph are HUVEC 100K: single culture, 1:1, 1:2, 1:5, 1:10, 1:20, 1:50; HUVEC 100K+ TNF-alpha: single culture, 1:1, 1:2, 1:5, 1:10, 1:20, 1:50; HUC single culture; and HUC single culture+TNF-alpha. This figure indicates that CD362+ stromal stem cells reduce inflammation.

FIG. 13B shows a graph depicting a decrease in TNF-alpha induced VCAM-1 in endothelial cells incubated with mesenchymal stem cells. The graph shows incubation in ratios of 1:1, 1:2, 1:5, 1:10, 1:20, and 1:50, as well as single culture of mesenchymal stem cells and endothelial cells. The bars from left to right in this graph are HUVEC 100K: single culture, 1:1, 1:2, 1:5, 1:10, 1:20, 1:50; HUVEC 100K+TNF-alpha: single culture, 1:1, 1:2, 1:5, 1:10, 1:20, 1:50; HUC single culture; and HUC single culture+TNF-alpha. This figure indicates that CD362+ stromal stem cells reduce inflammation.

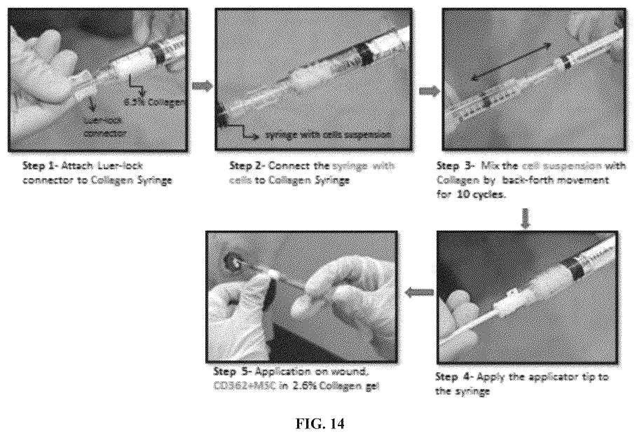

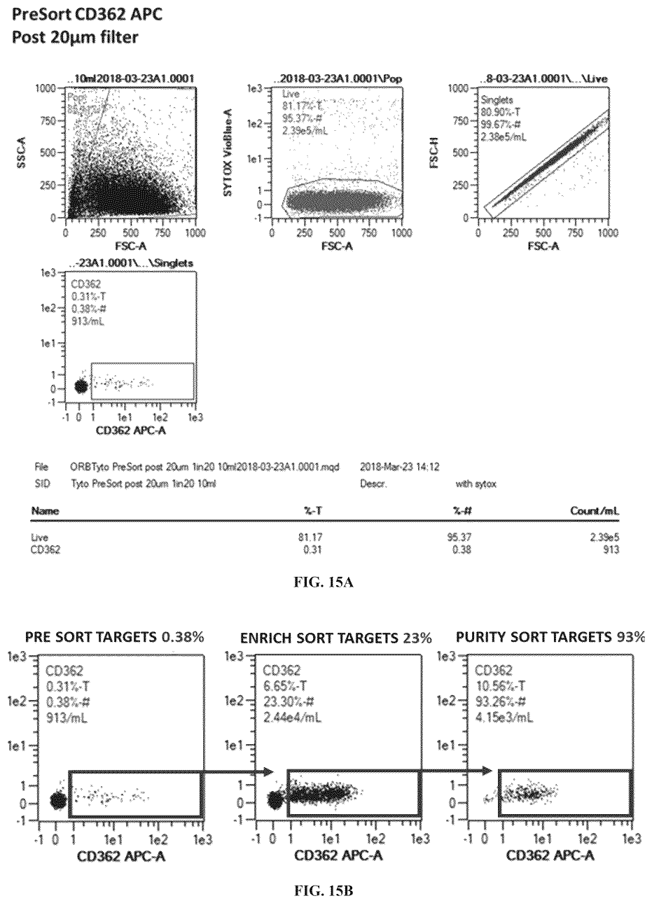

FIG. 14 shows photographs depicting preparation and administration of stromal stem cells for wound treatment. The panels are read in a clock-wise fashion starting with the top left panel. The first panel shows step 1, attaching a luer-lock connector to the syringe with collagen. The second panel shows step 2, connecting a second syringe with cells to the luer-lock connecter connecting two syringes. The third panel shows step 3, mixing the cells with the collagen by back and forth movement between the syringes for at least 10 cycles. The fourth panel shows the step 4, attaching the applicator tip to the syringe with the cell-collagen mixture. The last panel shows step 5, applying the cell-collagen mixture to a wound. This figure demonstrates an exemplary method of preparing stromal stem cells for topical administration.

FIG. 15A shows representative gating strategy for population analysis and CD362+ (SDC2+) target identification (population gate→viability gate (sytox blue exclusion)→singlets gate (doublets exclusion)→CD362 APC (target population)). This figure illustrates the gating strategy for purifying stromal stem cells for clinical use.

FIG. 15B shows identification of viable CD362+ target stromal cell population (highlighted in red square gate, percentage represent population after gating strategy applied), pre-sort CD362+ (0.38%), post first Tyto sort of CD362+ cells (enrich sort 23%) and final sort of CD362+ cells (purity sort 93%). This figure illustrates the purity of stromal stem cells purified for clinical use.

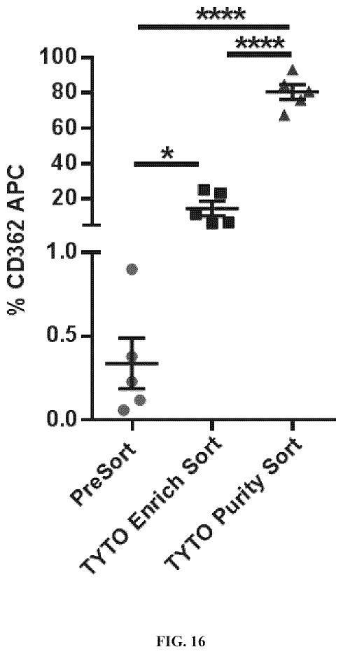

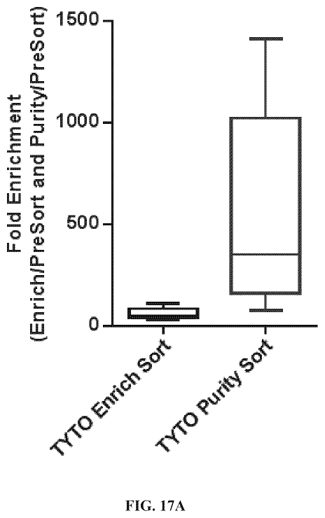

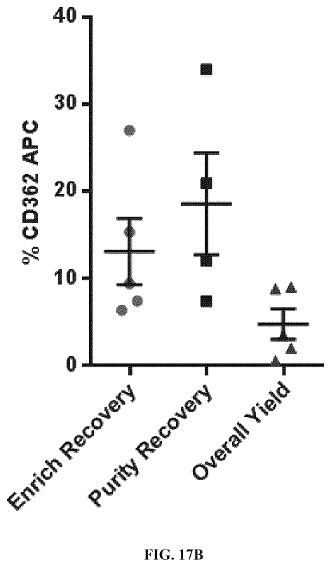

FIG. 16 shows average percentage population of CD362+ target stromal cell population in pre-sort (0.34±0.15%), enrich sort (14.52±4.1%) and purity sort (80.51±4.3%) (% mean±SEM n=5) after MACSQuant Tyto sorting. (*p≤0.05, ****p≤0.0001, ordinary one-way ANOVA, Holm-Sidak's multiple comparisons test). This figure illustrates the purity of stromal stem cells isolated for clinical use.

FIG. 17A shows average percentage population of CD362+ target stromal cell population in pre-sort (0.34±0.15%), enrich sort (14.52±4.1%) and purity sort (80.51±4.3%) (% mean±SEM n=5) after MACSQuant Tyto sorting. (*p≤0.05, **** p≤0.0001, ordinary one-way ANOVA, Holm-Sidak's multiple comparisons test). This figure illustrates the fold enrichment achieved in isolating stromal stem cells for clinical use.

FIG. 17B shows average percentage recovery of targets CD362+ population after enrich sort (13.1±3.8%), purity sort (18.58±5.9%) and final the overall yield of target CD362+ stromal cells from the starting population (4.8±1.8%) (% mean±SEM n=4-5). This figure illustrates the recovery of stromal stem cells in the isolation for clinical use.

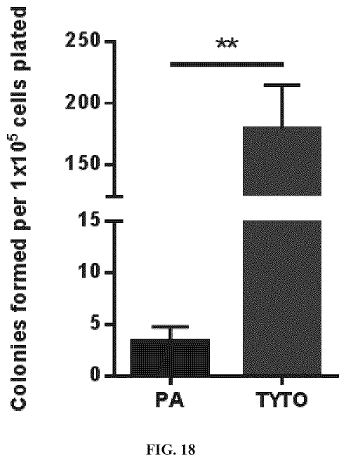

FIG. 18 shows CFU-f analysis of colonies formed from Tyto sorted (purity sort) CD362+ stromal (179.3±35.71 colonies per 1×105 cells plated) compared to standard plastic adherent (PA) unsorted cell fraction (3.4±1.4 colonies per 1×105 cells plated), (mean±SEM, n=3). (**p≤0.01, unpaired student t test). This figure illustrates the growth potential of stromal stem cells isolated for clinical use.

### Definitions

“Mesenchymal stem cells”, “stromal stem cells”, “SDC2+ stromal stem cells”, or “ORBCEL-C™”, used interchangeably are SDC2+ cells isolated from umbilical cord blood or bone marrow having therapeutic properties such as treating inflammatory diseases, such as inflammatory liver diseases, and wounds, such as non-healing wounds.

“SDC2,” also known as syndecan-2, CD362, S2, or fibroglycan, refers generally herein to the SDC2 polypeptide encoded by the SDC2 locus. Syndecan-2, or ‘the SDC2 protein’ or simply SDC2, is a transmembrane type I heparin sulfate proteoglycan. Additional synonyms for syndecan-2, aside from ‘the SDC2 protein’ or SDC2, include HSPG, CD362, HSPG1, and SYND2. Generally, as used herein SDC2 refers to the protein or a recognizable fragment thereof unless otherwise indicated, for example by reciting ‘the SDC2 gene,’ ‘the SDC2 transcript,’ ‘an SDC2 antibody.’ An SDC2 fragment refers to any set of consecutive residues of SDC2 that uniquely or recognizably map to the SDC2 polypeptide sequence. In some cases an SDC2 fragment retains some or all activity of the SDC2 protein, or acts as an inhibitor of full length or native SDC2. SDC2 also occasionally refers informally herein to the locus or gene encoding the SDC2 protein. In the event that one of skill in the art is unable to distinguish an SDC2 reference, it is presumed that the term is used herein in reference to the protein or polypeptide rather than to the gene, transcript, or an antibody raised against or binding to SDC2. There is a family of syndecan proteins in mammals. SDC2 is used alternately in reference to a mammalian syndecan-2 or to human SDC2 specifically. In the event that one of skill in the art is unable to distinguish an SDC2 reference, it is presumed that the term is used herein in reference to the human protein or polypeptide.

The terms “recipient”, “individual”, “subject”, “host”, and “patient”, are used interchangeably herein and in some cases, refer to any mammalian subject for whom diagnosis, treatment, or therapy is desired, particularly humans. “Mammal” for purposes of treatment refers to any animal classified as a mammal, including humans, domestic and farm animals, and laboratory, zoo, sports, or pet animals, such as dogs, horses, cats, cows, sheep, goats, pigs, mice, rats, rabbits, guinea pigs, monkeys etc. Sometimes, the mammal is human.

As used herein, the terms “treatment,” “treating,” “ameliorating a symptom,” and the like, in some cases, refer to administering an agent, or carrying out a procedure, for the purposes of obtaining an effect. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of a tumor in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease. Treating may refer to any indicia of success in the treatment or amelioration or prevention of an cancer, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms is based on one or more objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with cancer or other diseases. The term “therapeutic effect” refers to the reduction, elimination, or prevention of the disease, symptoms of the disease, or side effects of the disease in the subject.

The terms “pharmaceutically acceptable”, “physiologically tolerable” and grammatical variations thereof, as they refer to compositions, carriers, diluents and reagents, are used interchangeably and in some cases, represent that the materials are capable of administration to or upon a human without the production of undesirable physiological effects to a degree that would prohibit administration of the composition.

A “therapeutically effective amount” in some cases means the amount that, when administered to a subject for treating a disease, is sufficient to effect treatment or ameliorate a symptom of that disease.

As used herein, the term “about” a number refers to a range spanning that from 10% less than that number through 10% more than that number, and including values within the range such as the number itself.

As used herein, the term “comprising” an element or elements of a claim refers to those elements but does not preclude the inclusion of an additional element or elements.

“Inflammatory Liver Disease”, as used herein, refers to diseases causing inflammation of the liver including but not limited to autoimmune hepatitis, primary biliary cirrhosis, and primary sclerosing cholangitis.

“Non-healing wounds”, “wounds”, “open wounds”, “non-self-healing wounds”, “dermal wounds”, and “ulcerative wounds”, as used interchangeably herein, refer to chronic wounds that do not heal in an orderly set of stages and in a predictable amount of time. Wounds that fail to heal within three months are generally considered non-healing wounds. Non-healing wounds include but are not limited to diabetic wounds, venous ulcers, and pressure ulcers.

“Salve” and “Ointment” used interchangeably herein refer to formulations designed for topical administration to treat one or more skin ailments.

“About” a number, as used herein, refers to range including the number and ranging from 10% below that number to 10% above that number. “About” a range refers to 10% below the lower limit of the range, spanning to 10% above the upper limit of the range.

### EXAMPLES

The following examples are given for the purpose of illustrating various embodiments of the invention and are not meant to limit the present invention in any fashion. The present examples, along with the methods described herein are presently representative of preferred embodiments, are exemplary, and are not necessarily limitations on the scope of the disclosure. Changes therein and other uses which are encompassed within the spirit of the invention as defined by the scope of the claims are consistent with the disclosure herein.

### Example 1: Preparation of Stromal Stem Cells for Therapeutic Use

Stromal stem cells for therapeutic use were isolated from human umbilical cord tissue (UCT) by selecting for cells expressing CD362 (SDC2). These cells were shown to have a higher proliferative capacity compared to bone marrow derived mesenchymal stem cells (FIG. 2). This represents a significant increase in the potential cell yield available for therapeutic dosing from each cord used. By passage 7, CD362+ (SDC2+) mesenchymal stem cells have undergone >5 population doublings more than BM-MSC, which equates to a >30 fold increase in cell yield over marrow MSC protocols in manufacturing terms.

### Example 2: Preclinical Toxicology of SDC2+ Mesenchymal Stem Cells

A 90-day outsourced good laboratory practice (GLP) IV toxicology study of a single IV administration of ORBCEL-C™ (SDC2+ mesenchymal stem cells) to NU/NU nude mice (Nu-Foxn1nu) was performed. This was a single dose toxicology study (90 day duration) of SDC2+ mesenchymal stem cell product (ORBCEL-C™) in genetically immunodeficient mice.

Animals were weighed and systematically assigned to groups in the week prior to test administration. Males and females were randomized separately. After randomization, the mean body weight for each group by sex was not statistically different. The injection site was aseptically prepared and a dose of 1×10\hat{ }6 of anti-362 antibody-selected bone marrow mesenchymal stem cells (BM-MSC) and umbilical cord mesenchymal stem cells (UCT-MSC) made up to a total volume of 0.2 ml was administered intravenously via the tail vein of the 20 Nu/Nu mice at Day 0. Animals were terminated at Day 90. General health and clinical signs of toxicity were monitored prior to and 1 hour post-infusion. Non-fasting body weights were measured at Day 0 and weekly thereafter. Fasting body weight was measured prior to blood collection and necropsy at day 90. Hematology, coagulation and clinical chemistry parameters were tested from the blood collected.

At day 90, necropsy revealed no patterns of treatment related histological findings in systemic tissue and organs. There were no gross lesions identified and all tissues were macroscopically normal. No effect on weekly body weight or appetite was noted. There were no meaningful clinical findings for hematology, coagulation and clinical chemistry. Early deaths were not related to the administration of MSC.

### Example 3: Stromal Stem Cells Reduce Liver Inflammation

Three animal models of liver disease were used to investigate the efficacy of an infusion of human CD362+UCT-MSC versus unselected UCT-MSC on treating liver inflammation. These models were chosen to recapitulate in part important pathogenic pathways to liver inflammation. The pre-clinical models used included Mdr2KO/FVB mice, Ova-bil mice, and carbon tetrachloride mice. The Mdr2KO/FVB mouse is a chronic toxic bile acid induced biliary inflammation model commonly used to mimic experimental primary sclerosing cholangitis (PSC). Mice were administered either 2.5×10\hat{ }5 or 1×10\hat{ }6 MSC as a single infusion. The Ova-bil mouse model is a novel allo-immune model of antigen-specific induction of liver injury. These mice have an over-expression of ovalbumin in the biliary epithelium in their liver which is utilized to induce alloimmune liver injury after adoptive transfer of ova specific CD8+ T cells (isolated from OT1 transgenic mice) and CD4+ T cells (isolated from OT2 transgenic mice). The ova-specific CD4+ and CD8+ T cells migrate to the liver where they proliferate and induce necro-inflammatory damage. These mice were administered a dose of 5×10\hat{ }5 MSC. Carbon tetrachloride (CCl4) mice are a toxin induced acute liver injury mouse model. Mice were administered either 2.5×10\hat{ }5 or 1×10\hat{ }6 MSC as a single infusion.

There were no adverse events following administration of ORBCEL-C™ in all three animal models. Improvement was observed in various measures of liver function following MSC infusion including: improvement in serum ALT and ALP values (see FIG. 3A, FIG. 3B, and FIG. 3C); qualitative and quantitative decrease in histological inflammation and injury (see FIG. 4A and FIG. 4B); and quantitative analysis of lymphocytic infiltrate from whole liver digest (see FIG. 5A, FIG. 5B, FIG. 5C, and FIG. 6). Administration of CD362+UCT-MSC in MDR2KO/FVB mice resulted in the polarization of macrophages from the proinflammatory phenotype (M1) to anti anti-inflammatory phenotype (M2). This result was significant and was greater than when unselected MSC were used and control (see FIG. 7).

### Example 4: Immunosuppression of Human Cells by Stromal Stem Cells

CD362+UCT-MSC were co-cultured with anti CD3/CD28 stimulated CD4+ T cells isolated from peripheral blood of healthy volunteers for 5 days. Results showed that MSC significantly reduced CD4+ T cell proliferation compared to control (see FIG. 8). This effect was seen even at MSC: T cell ratio of 1:200.

Stimulated PBMCs isolated from patients with PSC were co-cultured with various ratios of CD362+UCT-MSC for 5 days and the effect on CD4+ T cell proliferation was investigated. Results showed that CD362+UCT-MSC reduced CD4 T cell proliferation effectively (see FIG. 9) compared to control (PBMCs only). This effect was noted even at lower concentrations of CD362+UCT-MSC i.e. 1:64.

Further work has been done to study immunogenicity of CD362+UCT-MSC (ORBCEL-C™) and their immunomodulatory potential following exposure to various stimuli e.g. IFN [symbol] . CD362+UCT-MSC were shown to be less immunogenic than BM-MSC as demonstrated in FIG. 10A and FIG. 10B, there was reduced susceptibility to CD8+ T cell and NK cell cytotoxicity. In addition CD362+UCT-MSC pre-treated with IFN [symbol] resulted in greater reduction in CD4+ T cell proliferation compared to MSC alone (see FIG. 11). These demonstrate the impact of the inflammatory environment by injected MSC in vivo.

### Example 5: Treatment of Acute Lung Injury by Stromal Stem Cells

The efficacy of ORBCEL-C in a Sprague Dawley (SD) rat model of E. coli-induced acute lung injury was tested. Briefly, rats were anesthetized and dosed intra-tracheally with 2×109 E. coli E5162 (serotype: O9 K30 H10) to induce an inflammatory lung injury. Within 1 hr of injury, 4 million (10M cells per kg) xenogenic human ORBCEL-C™ (or phosphate-buffered saline solution (PBS)) were delivered intravenously and the animals followed for 48 hrs. At 48 hrs, rats were intubated, the lungs were mechanically ventilated with 30% O2-systemic arterial blood pressure and peak airway pressure were continually measured. Notably, FIG. 12 demonstrates a significant improvement on lung oxygenation induced by ORBCEL-C™—over 7 distinct series of pre-clinical studies (placebo n=49; ORBCEL-C™ n=56)—where each data-point represent a group of 8 E. coli injured rats.

### Example 6: Reduction in Endothelial Cell Inflammation by Stromal Stem Cells

BM-MSC ORBCEL-M™ and UCT-derived ORBCEL-C™ were tested in an in vitro model of endothelial cell inflammation/activation—where human umbilical cord vein-derived endothelial cells—HUVECs—are stimulated with TNF α to induce the leukocyte adhesion proteins ICAM1 and VCAM1. Notably, in FIG. 13A and FIG. 13B, they demonstrate that the co-culture of ORBCEL-C™ with cytokine-stimulated HUVECs significantly reduced TNF α-stimulated ICAM1 and VCAM1, whereas co-culture with marrow-derived ORBCEL-M did not reduce endothelial cell activation.

### Example 7: Preparation of Stromal Stem Cell Compositions for Intravenous Administration

ORBCEL-C™ is derived from the ex vivo expansion of enriched CD362+ MSC isolated from human umbilical cord tissue (ORBCEL-C™). Cells are umbilical cord tissue-derived mesenchymal stromal cells, enriched by their expression of OBR1 (CD362/SDC2), by magnetic associated cellular sorting.

A weight based dosing regime is employed. Patients are recruited to a particular dose level i.e. either 1.0 (Dose level A), 2.5 (Dose level B) or 0.5 (Dose level C)×10\hat{ }6 cells/kg bodyweight.

The MSC cells are stored in CryoMACS bags (Miltenyi) by NHSBT in the vapor phase of liquid nitrogen at temperatures of <−150° C. All storage vessels are fitted with alarms that monitor temperature 24/7 and alert a member of staff on-call should a problem arise out of hours. Personal protective equipment such as lab coat and cryogenic gloves must be worn when handling the cryopreserved cells. Cells have been shown to remain viable when stored at less than −150° C. for at least 5 years. The cryopreservation and storage is validated to ensure the cell phenotype and viability is maintained during the cryopreservation and transport process. The cells are cryopreserved using a controlled rate freezer with a standard temperature reduction protocol. Bags of cells are transferred to dry shippers for transfer to the clinical unit. These are also validated to carry cells at <−150° C. in the vapor phase of liquid nitrogen. The transport containers are also fitted with temperature monitors and alarms.

The cryopreservation process has been tested to show that cell viability is maintained above 90% using the 7AAD stain by flow cytometry. Cells undergoing typical transportation to a clinical site and return to the manufacturing site in a dry shipper have also been shown to maintain viability above 90% and grow normally in culture after being thawed.

Cryopreserved ORBCEL-C™ are delivered directly from the manufacturing site (NHS Blood and Transplant in Birmingham) to the recruiting center in CryoMACS bags using a validated container (dry shipper) at less than −150° C. in the vapor phase of liquid nitrogen. Please refer to the ATMP Manual for detailed information on administration. In summary, ORBCEL-C™ are administered to the patient within 48 hours of release directly and within 15 minutes after thawing. When the patient is prepared for the infusion, ORBCEL-C™ is thawed—one bag at a time—by placing each bag into a water bath close to the patient's bedside. Each CryoMACS bag containing ORBCEL-C™ is removed from the water bath as the last crystals thaw and the bag is checked for any damage. The overwrap bag is removed carefully with scissors after thawing, once it has been established no damage has occurred to the primary bag. Subsequent bags should not be thawed until the previous bag infusion is nearing completion. The water bath is filled with sterile saline or water for each patient and maintained at a temperature of 37° C. Each bag ID is double checked against the patient's ID by two operators and these checks are recorded with signatures by the operators. Operators must wear thermal protection gloves and safety goggles when removing bags from the dry shipper and placing them in the water bath. Cells from a damaged bag are be used.

### Example 8: Amelioration of Liver Inflammatory Disease Using Stromal Stem Cell Compositions

Herein is an adaptive, single arm, multi-center, phase IIa multi-disease clinical trial. It is designed to: i) determine dose safety of ORBCEL-C (selected Mesenchymal stromal cells derived from human umbilical cord and ii) evaluate treatment activity through assessment of biomarkers.

This trial has two main stages. Stage 1 determines the maximum tolerated dose that can be administered by observing for occurrence of dose limiting toxicity (DLT). Stage 2 uses the maximum tolerated dose found in stage 1 and further determines safety and activity outcomes of ORBCEL-C.

TABLE 1
Clinical interventions in Inflammatory Liver Disease

| Arms | Assigned Interventions |
|---|---|
| Experimental: PSC patients | Biological: Orbcel-C Selected |
| Selected Mesenchymal Stromal Cells | Mesenchymal Stromal Cells |
| (dose selection and efficacy)-Orbcel-C. | derived from human umbilical |
| dose selection, combination of 0.5, 1.0, | cord |
| 2.5 million cells/kg (3 dose levels). | |
| IV single-infusion. | |
| Experimental: AIH patients | Biological: Orbcel-C Selected |
| Selected Mesenchymal Stromal Cells | Mesenchymal Stromal Cells |
| (dose selection and efficacy)-Orbcel-C. | derived from human umbilical |
| dose selection, combination of 0.5, 1.0, | cord |
| 2.5 million cells/kg (3 dose levels). | |
| IV single-infusion. | |

The primary objective of Stage 1 is to determine the maximum tolerated single intravenous infusion dose of ORBCEL-C over a 14-day reporting period to take forward to Stage 2 of the clinical trial (study). All patients who have been recruited to and completed the 14-day reporting period in stage 1 continue are evaluated for outcomes as per Stage 2.

The primary objectives of Stage 2 are to investigate whether a single intravenous infusion of ORBCEL-C in patients with PSC and AIH is safe and tolerated over the period of trial follow up (up to 56 days). It is determined whether ORBCEL-C reduces serum alkaline phosphatase (ALP) in patients with PSC. This is a non-invasive biochemical surrogate of clinical outcomes in PSC. In patients with AIH, it is determined whether ORBCEL-C reduces serum alanine aminotransferase (ALT). This is a non-invasive biochemical surrogate marker of hepatic inflammatory activity and outcomes in AIH.

The secondary objectives of Stage 2 are to investigate whether a single intravenous infusion of ORBCEL-C elicits a change over the duration of the study after treatment in patients with PSC and AIH, including (i) liver biochemistry and function, immunoglobulin G concentrations (in AIH patients) and composite risk scores; (ii) non-invasive clinical markers of fibrosis; (iii) patient quality of life (QoL); and (iv) severity of co-existent IBD in patients with PSC.

Further exploratory research objectives of the trial determine whether MSC infusion modulates the immune response by measuring whether treatment elicits a change in patients with PSC and ATH including (i) markers of immune activation including immunoglobulin values and C-reactive protein concentration; (ii) markers of biliary injury including total bile acid levels; (iii) circulating inflammatory cells profile this includes phenotypic expression of T regulatory cells (Tregs) a common mechanistic primary endpoint; (iv) endothelial cell activation markers such as VAP-1 and ICAM1; (v) serum cytokine, chemokine, microRNA and RNA expression profiles

Eligibility criteria include: (i) ages Eligible for Study: 18 Years to 70 Years; (ii) sexes Eligible for Study: all; (iii) accepts healthy volunteers: no.

Inclusion criteria for PSC patients include: (1) age≥18, ≤70 years old at visit 1 (screening); (ii) diagnosis of PSC at visit 1 (screening) as evidenced clinically by: chronic biochemical cholestasis (elevated serum alkaline phosphatase (ALP) above the upper limit of normal (ULN) and/or gamma-glutamyl transpeptidase (GGT) above the ULN)>6 months duration and Radiological and/or histological evidence of clinically documented PSC Serum ALP)≥1.5 ULN at visit 1 (screening) Serum ALP value at Visit 2 within +/−25% of ALP value at visit 1.

Inclusion criteria for AIH patients include: (i) age≥18, ≤70 years old at visit 1 (screening); (ii) established pre-existing clinical diagnosis of AIH confirmed by clinical expert review consistent with the simplified IAIHG criteria (exemplary criteria is available at the website, mdcalc.com/simplified-scoring-autoimmune-hepatitis-aih/) and must include history of a liver biopsy reported compatible with AIH; (iii) active AIH defined by ALT≥1.5 ULN; (iv) serum ALT must be above ≥1.5 ULN at both screening (visit 1) and visit 2; (v) at visit 2, it should be confirmed that a patient does not meet any of the exclusion criteria; (vi) patients must be on standard-of-care AIH treatment for ≥24 weeks-this includes any AIH therapy except biologics; (vii) stable doses of immunosuppression for a minimum period of 4 weeks at the time of screening, and no planned change in immunosuppression for the course of the trial.

Generic exclusion criteria that apply to both patients with PSC and AIH include: (i) creatinine>133 μmol/L or being treated with renal replacement therapy at the time of Visit 1 (screening); (ii) AST or ALT>10×ULN; ALP>10×ULN; (iii) platelets<50×109/L; Total Bilirubin>2×ULN; INR>1.3 (in the absence of concomitant use of Warfarin or equivalent anti-coagulant therapy); (iv) albumin<35 g/liter; (v) hemoglobin<10 g/dl; (vi) past or present evidence of decompensated chronic liver disease; (vii) radiological or clinical evidence of ascites; (viii) hepatic encephalopathy; and (ix) endoscopic evidence for portal hypertensive bleeding.

Exclusion criteria specific to patients with PSC include: (i) documented alternative etiology for sclerosing cholangitis (i.e. secondary sclerosing cholangitis); (ii) dominant (as determined by Investigator) alternative chronic or active liver injury other than PSC at the time of visit 1 (screening); (iii) patients with possible overlap syndrome with AIH are excluded from the PSC cohort if the Investigator considers AIH as the dominant liver injury; (iv) ALP>10×ULN; (v) evidence of cholangitis within 90 days of visit 1 (screening); (vi) documented evidence of cholangitis by physician; (vii) need for any antibiotics for presumed cholangitis; (viii) any patient taking prophylactic antibiotics to combat recurrent cholangitis; (ix) presence of percutaneous biliary drain, or internal biliary stent; (x) diagnosed hepatocellular carcinoma, cholangiocarcinoma, or high clinical suspicion thereof; and (xi) dominant stricture clinically suspicious of cholangiocarcinoma (as determined by Investigator).

Exclusion criteria specific to patients with AIH include; (i) dominant (as determined by Investigator) alternative chronic or active liver injury other than AIH at the time of visit 1 (screening); (ii) patients with possible overlap syndrome with PSC are excluded from the AIH cohort if the Investigator considers PSC as the dominant liver injury; AST or ALT >10×ULN; (iii) patients on a prednisolone dose of >20 mg at the time of screening; and (iv) diagnosed hepatocellular carcinoma or cholangiocarcinoma or high clinical suspicion thereof.

In Stage 1, the short-term safety of a single peripheral vein infusion of different doses of ORBCEL-C™ is assessed by occurrence of DLT with the aim being to select with the aim being to select the maximum tolerated dose to take forward to Stage 2. If the safety of the entry dose (dose level A: 1×10\hat{ }6 cells/kg body weight) is confirmed, the safety of the higher dose (dose level B: 2.5×10\hat{ }6 cells/kg body weight) is assessed. If the dose level A infusion dose is deemed unsafe by the occurrence of DLTs, then the safety of the higher dose is not be examined, but a lower dose (dose level C: 0.5×10\hat{ }6 cells/kg body weight) is tested.

The maximum tolerated dose determined in Stage 1 is selected for recruitment of subsequent patients in Stage 2 (applies to both PSC and AIH cohorts). All the subsequent patients recruited in Stage 2 receive a single peripheral vein intravenous infusion of the selected dose. A weight based dosing regime is employed. Two bag units (Bag A and Bag B) are available for use. Whole bags must be used, by instruction of NHSBT, and the calculated doses for any given weight, reflect this. Bag Assignment of Doses: Bag A 80×10\hat{ }6 cells and Bag B 40×10\hat{ }6 cells.

Patients need to fast on the day of infusion (serum bile acid levels are taken fasting). Once bloods have been taken, the patient can eat and drink,

All patients are administered chlorpheniramine (which is regarded as a non-Investigational Medicinal Product for the purposes of this trial) 10 mg by peripheral IV bolus, given approximately 30 minutes prior to infusion.

ORBCEL-C™ MSC infusion via large-bore (green/18G or larger) cannula by Y-connector giving-set. Each bag of MSC is given over 10-15 minutes, and given serially via the same giving set. The other arm of the y-connector is attached to a 250 ml bag of normal saline, which is infused over the total anticipated duration of the MSC infusion. The end of the last bag of the MSC marks the end of the MSC infusion. On completion of the MSC infusion, the remaining cells are flushed with 250 ml of normal saline (over 30 mins) which is infused via the same arm of the y-connector as the MSC.

Peri-infusion vital signs monitored include 5 minutes pre-infusion; every 15 minutes throughout the infusion; then at 30, 60, 120 and 240 minutes post cessation. Standard bloods and exploratory research bloods are taken 4 hours after completion of infusion.

### Example 9: Preparation of Ointments and Salves

REDDSTAR ORBCEL-M is produced in LUMC, Leiden and combined with 6.5% collagen from Collagen Solutions on site in Copenhagen. Both products are shipped separately to Bispebjerg hospital and mixed by syringe mixing in a biosafety cabinet to produce a therapeutic product (REDDSTAR ORBCEL-M in 2.6% collagen,) for administration to the patient.

Cell dose: A 30×10\hat{ }6 REDDSTAR ORBCEL-M (CD362+ MSC) fixed dose per patient which is initially produced in LUMC and preserved at −80° C. in freezing medium (HSA/DMSO) while release testing is performed. When a request for cell product comes through from Copenhagen (treatment day—11 days), LUMC thaws the cells and expands, centrifuges, and resuspend the cells in 500 μl Hypothermosol (such as Hypothermosol described at the website, biolifesolutions.com/cgmp-biopreservation-media-products/hypothermosol/) in a luer lok syringe. Shipment is by World courier to Copenhagen for mixing with 350 μl of 6.5% collagen gel that has been previously manufactured by Collagen solutions in Scotland (such collagen is described at the website, collagensolutions.com/index.asp) and shipped to Copenhagen. Several batches of 6.5% collagen are stored on site in Copenhagen.

The two constituents of the product are transported to Zelo Phase I Unit, Bispebjerg Hospital, Denmark. Cells are released for administration to the patient. The two constituents are mixed in a biosafety cabinet and applied to the wound at the patients beside by a healthcare professional.

The protocol for mixing of the IMP is as per FIG. 14 below:
1. The 6.5% Collagen syringe is transported to Zelo Phase I Unit, Bispebjerg Hospital, Denmark and is stored at 4° C. REDDSTAR ORBCEL-M is manufactured according to GLP guidelines at Leiden and is released and then transported separately to Zelo Phase I Unit, Bispebjerg Hospital, Denmark. The treatment (mixing of MSC with collagen) is be prepared on the day of application to the wound, immediately before application.
2. When ready for application to wound, the syringe containing 0.35 ml 6.5% Collagen is fitted to a syringe containing 0.5 mL of the REDDSTAR ORBCEL-M cell suspension via a Luer Lok connector and is mixed by passing the syringe back and forth 10 times to mix, starting by pushing the collagen into the REDDSTAR ORBCEL-M syringe The end result is a suspension of REDDSTAR ORBCEL-M in a final concentration of 2.6% Collagen in the original 1 mL REDDSTAR ORBCEL-M cell syringe.
3. The REDDSTAR ORBCEL-M/collagen preparation is transported to the patient in a tamperproof container prior to administration

### Example 10: Treatment of Diabetic Wounds With Stromal Stem Cell Compositions

Described herein is a Phase 1b, open label, uncontrolled non-randomized single dose study to examine the safety of topically applied bone marrow derived allogeneic mesenchymal stromal cells (REDDSTAR ORBCEL-M) seeded in a collagen scaffold to patients with non-healing neuroischaemic diabetic foot wounds. The objective of the trial is to determine the feasibility and safety of topically administered allogeneic bone marrow-derived mesenchymal stromal cells (REDDSTAR ORBCEL-M) seeded in a collagen scaffold to patients with non-healing neuroischaemic diabetic foot wounds.

Principal inclusion criteria include: (i) age 18-80 years; (ii) Type 1 or Type 2 diabetes mellitus (with any kind or combination of pharmacological treatment for disease and/or complications to disease); (iii) HbA1c≤97 mmol/mol (≤11%); (iv) males or non-pregnant females; (v) understand trial information document; (vi) provide written informed consent; (vii) duration of (diabetic foot) wound>4 but <52 weeks; (viii) reduction of <50% area over 4 weeks despite standard care (standard care; off-loading, weekly debridement, dressings, orthotic); (ix) wound area with sharp debridement of ≥0.5 but ≤4.0 cm²; (x) clinically non-infected wound; (xi) Texas wound stage 1a, 1c or 2a; (xii) location of wound below malleolus; (xiii) affected limb toe pressure≥40 mmHg; or a Doppler waveform consistent with adequate flow in the foot (biphasic or triphasic waveforms); (xiv) an ankle-brachial systolic pressure index between 0.7 and 1.3; (xv) diagnosis of peripheral neuropathy using ADA guidelines (monofilament/vibration sensation/biothesiometer); (xvi) adhere to study visit protocol; and (xvii) adhere to offloading devices/orthotic.

Principal exclusion criteria include: (1) life expectancy of less than 12 months; (ii) patients with a definite diagnosis of any immunodeficiency disorder; (iii) viral hepatitis [patient must have negative hepatitis B surface-antigen (HBsAg) and hepatitis C antibody (HepCAb) test results obtained within 2 weeks prior to the Treatment Day (Day 1)]; (iv) active, uncontrolled connective tissue disease; (v) renal failure as defined by serum creatinine>220 μmol/L; (vi) liver function tests (e.g. AST, ALT) that are >2.0 times ULN; (vii) poor nutritional status as measured by serum albumin<30 mg/dL; (viii) active cancer or a history of cancer in the 5 years prior to signing the informed consent form (history of basal cell carcinoma is allowed); (ix) active wound infection (i.e. recent onset of erythema, edema, and increased temperature of the foot with normal radiographs); (x) Diabetic Charcot neuroarthropathy or other structural deformity that would prevent adequate off-loading of the study foot; (xi) treatment with any systemic corticosteroid immunosuppressive chemotherapeutic agent, antiviral, or previous/current radiation therapy to lower extremity treated within 30 days prior to signing the informed consent form; (xii) having received another investigational drug or biologie within 30 days prior to signing the informed consent form or currently participating in an investigational drug or biologic study; (xiii) Haemoglobin A1c (HbA1c) test result of >11% (>97 mmol/mol) documented at the screening visit; (xiv) wounds caused primarily by untreated vascular insufficiency, or where patients are primarily eligible for vascular intervention to promote wound healing; (xv) wounds with an etiology not related to diabetes; (xvi) more than three wounds on the target lower extremity; and (xvii) the wound studied not anatomically distinct from another wound(s) (separated by <1 cm from another wound or would interfere with standard of care treatment of another wound (only one single wound per one study subject is treated in this study; (xviil) wounds which decrease in area by >50% during the screening 4-week run-in period; (xix) ulcers with underlying osteomyelitis on the leg with the wound treated; (xx) patients presenting with the clinical characteristics of cellulitis at the wound site (suppurative inflammation involving particularly the subcutaneous tissue, often mild erythema, tenderness, malaise, chills and fever); (xxi) revascularization surgery on the leg with the wound treated≤8 weeks prior to signing the informed consent form; (xxii) surgery to lengthen Achilles tendon on the leg with the wound treated≤8 weeks prior to signing the informed consent form; (xxiii) necrosis, purulence, or sinus tracts that cannot be removed by debridement on foot treated; (xxiv) received dermal substitute or living skin equivalent (Leukopatch within 30 days prior to signing the informed consent form; (xxv) received prior PDGF-BB (Regranex®/becaplermin) therapy within 30 days prior to signing the informed consent form; and (xxvi) has known history of clinical sensitivity reactions to products of bovine origin or to the primary or secondary dressings used in the trial.

Primary end point is the number and severity of serious adverse events considered related to treatment with study drug (mesenchymal stromal cells; REDDSTAR ORBCEL-M), measured from time of application of mesenchymal stromal cells until end of study visit.

Secondary end points include: (i) safety (Adverse events (serious or non-serious)) and (ii) efficacy: a) ulcer healing trajectories over time as assessed by plots of percentages of ulcer closure versus assessment of time; b) time to complete ulcer closure (as defined as Treatment Day 1 to the first visit when closure is documented); c) absolute and percent changes in ulcer area from baseline at weekly intervals through week 12; d) durability of ulcer closure as measured at 4 week intervals for 12 weeks from date of ulcer closure. Measurement of secondary endpoint is: (i) for safety: from time of application of mesenchymal stromal cells until end of study visit; and (ii) for efficacy: a) from time of application of mesenchymal stromal cells until 12 weeks after (or until ulcer closure), measured at weekly intervals; b) from time of application of mesenchymal stromal cells until ulcer closure, measured at weekly intervals; c) from time of application of mesenchymal stromal cells until 12 weeks after (or until ulcer closure), measured at weekly intervals; and d) from time of ulcer closure until 12 weeks after (or until re-opening of ulcer), measured at 4 week intervals.

### Preparing the Wound for Treatment

The wound is debrided on day −39 to start the screening 4-week run-in period and on day −11. Ulcer area measurements are made on Day −11 following ulcer debridement to confirm that the ulcer area is appropriate for the study. If it is medically necessary the ulcer is debrided prior to ulcer assessment. Since the goal of ulcer site preparation is to convert the ulcer environment from that of a chronic wound to that of an acute wound, it is important to treat the ulcer site as soon as possible after debridement/scoring of the wound bed to take advantage of the influx of blood into the ulcer. Sharp surgical debridement is performed to remove all necrotic soft tissue, hyperkeratotic wound margins, bacterial burden, cellular debris, sinus tracts, fistulae, undermined borders, and callus to produce viable wound margins and a clean ulcer site. If debridement is not necessary then the ulcer site and margins are lightly scored to create the small influx of blood that is needed to facilitate ulcer healing. Mechanical, enzymatic, biological, or autolytic debridement are not used in this study. Pressure is applied to stop bleeding. Silver nitrate is not used to stop ulcer site bleeding.

Ulcer area is measured using the acetate tracing method, photograph is taken, and using Silhouette device. Sterile saline is used to remove any debris left after debridement. The ulcer is irrigated with saline and debris is gently wiped from the ulcer with sterile gauze using care to minimize mechanical trauma at the site. A moist (sterile saline) sterile gauze is used to keep the ulcer moist until the study treatment is administered; the ulcer should contain some blood to facilitate the effects of the study treatment. A dressing is used that will maintain a moist wound-healing environment, manage wound exudates, and protect the peri-ulcer skin (e.g. Opsite/Mefix). Sterile gauze is cut in the approximate size and shape of the ulcer, and placed over the ulcer. The dressing is wrapped with a non-adherent bandage (e.g. Mepilex® neutral foam) to secure the wound dressing and a white sock is placed on the foot so exudate can be visualized should it occur.

### Infection Control

Each time the ulcer dressing is changed, the ulcer is cleansed with saline and a moist sterile gauze pad. In addition, if infection of the ulcer is suspected, culture and sensitivity tests are performed and appropriate oral and/or intravenous antimicrobials (other than antivirals) are initiated to treat the infection. Patients may also receive systemic antimicrobial therapy for suspected or documented infections outside the treatment site.

### Weight Off-Loading Device

All patients wear a special weight off-loading orthopedic shoe (DH Aircast Walker) starting on Day −39 (the first day of the screening run-in period) through 2 weeks after ulcer closure, or Week 12 if the ulcer does not close.

### Treatment

The REDDSTAR ORBCEL-M cells in 2.6% collagen will be topically applied to the neuroischaemic diabetic foot wound. The dose administered is 10.6×10\hat{ }6 MSC per 3 cm² wound.

For treatment application on Day 1, the ulcer site is prepared as per the instructions in section above (i.e. debridement if medically necessary [if debridement is not necessary, the ulcer site and margins should be lightly scored to create the small influx of blood that is needed to facilitate ulcer healing], ulcer measurement, irrigate with saline, and make sure that the treatment site is moist).

The study personnel will use the ulcer area measurement obtained from measurement on the Treatment Day I to calculate the total number of cells and volume of REDDSTAR ORBCEL-M/Collagen that is applied topically to the ulcer. This will be recorded and the cells will be mixed as described herein.

The study personnel will mix the collagen and the REDDSTAR ORBCEL-M suspensions in the mixing device provided and apply the required volume (10.6×10\hat{ }6 MSC/3 cm² wound) of REDDSTAR ORBCEL-M/collagen as a continuous film over the entire ulcer surface area, to the margins. The subject treated with REDDSTAR ORBCEL-M/collagen will in addition receive standardized care at weekly intervals.

The patients continue to receive treatment for diabetes as needed following the prescription(s) of his/her regular attending physician. If possible, the patient maintains the same therapy received prior to study enrolment through the end of study visit. Concomitant medications are kept to a minimum during the study. However, if they are considered necessary for the patient's welfare and are unlikely to interfere with the study treatment, they are given at the discretion of the Investigator.

Guidelines regarding concomitant medications that may affect ulcer closure as follows: (i) no concomitant therapy may be applied to the affected foot starting from Day −39 through the end of study visit (e.g., antimicrobials, steroid cream); (ii) systemic antibiotics may be taken (if deemed necessary by the Investigator) and are recorded in the EDC system; (iii) concomitant medications that might interfere with the study treatment are not permitted (e.g. oral corticosteroids, immune-suppressants, chemotherapy and antiviral drugs). Treatment with any other investigational drug or device is not permitted during the study.

### Example 11: Preparing SDC2+Stromal Stem Cells From Human Umbilical Cord

Reagents and materials needed include 70% isopropyl alcohol, (×1) Dulbecco's Phosphate Buffered Saline (Gibco), (×2) MEM Alpha with Glutamax, (110 mls) FBS, Stock Collagenase-1 (50 mg/ml), (500 ml) CliniMACs Buffer, CD362 APC, TYTO CARTRIDGE, TYTO BUFFER, Filters (100 μm), Pre-separation filters (20 μm), Penecillin-Streptomycin, aliquoted, FGF-2, aliquoted, Sytox Blue, 6-well culture plates, 12-well culture plates, 25 cm² culture flasks, 75 cm² culture flasks, and 175 cm² culture flasks.

Dissociation and Digestion proceeded as follows. Ensure temperature and cord details are recorded. Turn on incubator for Digestion Temp 37° C. Turn on the MACsQuant and Calibrate Machine (Please refer to appropriate SOP). Remove the umbilical cord from the shipping container using sterile forceps and place in a sterile 50 ml tube and seal. Use an empty 50 ml tube to zero balance scales. Then weigh the 50 ml tube containing the umbilical cord. Record weight of the tissue. Place the cord in a tissue culture dish containing 70% IPA and wash ˜30-40 sec. Rinse and remove to petri dish containing cold PBS. Remove any large clots using sterile forceps and clear cord of blood. Transfer the cord to fresh petri dish containing cold PBS. Divide the cord into equal length 5 g segments and return to the petri dish containing cold PBS. Calculate the number of 50 ml Falcon tubes needed for digestion assuming 5 g of tissue per tube, (e.g., Cord weight 3 g-6 g=1 tube, Cord weight 7 g-12 g=2 tubes, Cord weight 13-16 g=3 tubes, Cord weight 17-22 g=4 tubes, Cord weight 23-26 g=5 tubes, or Cord weight 27-32 g=6 tubes). Transfer 6.5 ml of cold Alpha MEM to each tube. Using a sterile forceps transfer a cord segment to a fresh petri dish plate and use a scalpel blade to manually dissociate the cord segment into small pieces (0.5-1 mm2). Keep washing segments free from any contaminating cord blood. After the dissociation is complete transfer the tissue (5 g/tube) to the 50 ml Falcon tube containing the 6.5 ml of Alpha-MEM media. Repeat steps 4.12 and 4.13 for each cord segment and until the cord is complete homogensied. After dissociation prepare Digestion Buffer: Number of Falcon Tubes×6 ml=X ml=______. Add X ml of media to a fresh falcon tube labelled Digestion Buffer. Number of Falcon Tubes×12.5 ml (final vol per tube)×2 (final conc per tube)=Y mg of Col-1 (Stock Col-1 50 mg/ml). Remove Y mg=(Y×1 ml/50)ml=Z ml=______. Add Z ml of Col-1 to the falcon tube labelled Digestion Buffer. Add an equal volume (6 ml) of Digestion Buffer to each homogenized tissue tube. Each tube contains 5 g of tissue, a final volume of 12.5 mls and a final concentration of 2 mg/ml Col-1. Place each tube on the Thermal Rocker laying on their side (make sure temperature reads 37° C. in incubator) secure tubes using tape or parafilm. Incubate for total of 45 mins. Set timer. After digestion run is complete neutralize each tube with 10 ml of cold Stop Solution (Alpha-MEM with 10% FBS). Centrifuge all tubes for 1 min @ 400 rcf @ 4° C. (Accel9, Decel9). Prepare and equal number of fresh falcon tubes to number of digestion tubes and place 100 μm cell strainer on each tube. Prime each filter with 1 ml of Stop Solution. Filter supernatant of sample through each 100 μm cell strainer into fresh 50 ml falcon tubes (pour supernatant) leaving the tissue pellet behind. Add 10 ml of Stop Solution to each digestion falcon tube containing the remaining tissue pellet, mix by tube inversion and centrifuge for an additional 1 min @ 400 rcf @ 4° C. (Accel9, Decel9). Filter supernatant of sample through each 100 μm cell strainer into fresh 50 ml falcon tubes already containing supernatant (pour supernatant). Add the remaining tissue to the filter (pour tissue). Rinse each falcon tubes out with 2 ml of Stop Solution and add to filters. Allow cells suspension to flow through. Discard filter (may contain tissue) and bring each falcon tube volume up to 50 ml with Stop Solution. Centrifuge each tube for 10 minutes @ 2000 rcf @ 4° C. (Accel9, Decel9). Transfer the supernatant from each tube into a fresh 50 ml Falcon tube and centrifuge for an additional 10 minutes @ 2000 rcf @ 4° C. (Accel9, Decel9). Prepare a fresh falcon tube with a 100 μm filter primed with CliniMACs Buffer (CliniMACs+1% FBS). Add 10 ml of CliniMACS Buffer to one tube and resuspend pellet transfer the cell suspension to the next tube and resuspend the pellet—repeat for all tubes and filter cell suspension into fresh falcon tube. Rinse each falcon tube with an additional 10 ml CliniMACs buffer and add to filtered cell suspension. Discard supernatant from each centrifuged tube and resuspend each cell pellet as described previously. Add the cell suspension to the already filtered cell suspension. Rinse the filter with fresh CliniMACs buffer to bring final volume of cell suspension to 50 mls. Resuspend the HUC Single Cell Suspension well. Take a 20 μl sample of HUC Single Cell Suspension and mix with buffer (1:10) in Eppendorf (200 μl). Use MACsQuant for count (Please refer to appropriate SOP). Login to appropriate account. Open HUC Instrument Settings. Open HUC Experiment Settings. Open HUC Analysis Template. Add Experimental and Sample Details. Add Sytox Blue to Eppendorf (1:50) and acquire 180 μl of sample. Record number of viable cells=______. Total Number of cells=Viable Cells×10 (Dilution Factor)×50 ml (Total Vol): ______. Centrifuge HUC cell suspension at 400 rcf @ 10 min @ 4° C. (Accel9, Decel9).

CD362 APC labelling of stromal stem cells proceeded as follows. Remove supernatant from HUC cell suspension. Re-suspend the sample in 900 μl CliniMACs buffer (up to a final concentration of ≤1.5×108 cells/900 μl, for example ≤1.5×108 cells/900 μl, ≤2.5×108 cells/1800 μl, and ≤3.5×108 cells/2700 μl). Volume Used: ______. Add 100 μl CD362 APC ab (1:10 dilution) per 900 μl of HUC cell suspension, for example, ≤1.5×108 cells/100 μl ab, ≤2.5×108 cells/200 μl ab, ≤3.5×108 cells/300 μl ab. Volume Used: ______. Incubate cell suspension with antibody for 30 min at 4° C. in the dark. Add CliniMACs buffer for final volume 50 mls and centrifuge at 400 rcf @ 5 min @ 4° C. (Accel9, Decel9). Add CliniMACs buffer for final volume 50 mls and centrifuge at 400 rcf for 5 min. Prime MACSQuant Tyto cartridge: 1. Load the cartridge into the priming fixture; 2. Orient the cartridge so the blue luer caps appear in a ∴ shape; 3. Place the cartridge into the side slot and the feet into the corresponding slots in the base; 4. Verify that the cartridge feet are also positioned in their corresponding slots, it should sit level in the fixture; 5. Load a 10 mL syringe with 1 mL Tyto Buffer; 6. Remove the blue input luer cap on the cartridge (right side) and insert the tip of the filter into the opening; 7. Expel enough Tyto Buffer into the input chamber so that the level reaches the stir propeller (˜500 uL); 8. Pull the syringe plunger out to its stop and attach the syringe back to the input luer lock; 9. While pressing on the left side of the cartridge, push the plunger all the way down to pressurize the chamber and flow Tyto Buffer through the cartridge the to the sort chamber. Hold the plunger until a visible volume of the liquid has been pushed through; 10. Remove the cartridge from the priming fixture; 11. Detach the syringe from the cartridge and pull the plunger out to its stop before re-attaching the syringe to the input luer; 12. Using the same syringe remove any remainder buffer from the input chamber; 13. Using a pipette remove the volume form the negative chamber; 14. Proceed with loading sample into the cartridge. Re-suspend cells in volume of Tyto Buffer 10-20×10e6 cells/ml (total of 4 mls). Prep filter (20 μm) placed into a capped 10 ml syringe with 500 μl of Tyto Buffer. Filter sample cell suspension and rinse falcon tube with an additional 4-6 ml of Tyto buffer for final concentration of 4-8×10e6cells/ml in a final vol of 8-10 mls and add to syringe via filter. NOTE: If cell concentration high ≥4×10c6cells/ml after reconstitution in 10 mls of Tyto Buffer load only 5 mls into cartridge at a time to reduce clumping. Once 4.5 ml has been sorted stop the sort and add the remaining cells suspension. Add cell suspension to primed Tyto Cartridge. Take a 10 μl sample of Pre-Sort Post Filter Single Cell Suspension and mix with buffer (1:20) in Eppendorf (180 μl). Add Sytox Blue to Eppendorf (1:50) and acquire 180 μl of sample. Record number of viable cells=______. Total Number of cells=Viable Cells×20 (Dilution Factor)×2 ml (Total Vol): ______. Remove 1×10\hat{ }6 cells for WT culture.

MACSQuant TYTO was run as follows. Turn on MACSQuant Tyto instrument by tapping the small orange screen. Launch MACSQuantify software and log into MACSQuant Tyto software. Locate identification sticker on the end of the cartridge and scan using barcode scanner. Confirm dialog by clicking on the button “Use cartridge”. Slide instrument door open. Orient the MACSQuant Tyto cartridge so that the Tyto owl image is in the upright position. Place the cartridge in the right hand side slot of the instrument stage. Do NOT push the cartridge downward. You will hear a click as the cartridge automatically locks into position. The door will close automatically. The handle will flash red and an alarm will sound 10 seconds before closure. Hold the door in place if more time is required for insertion of cartridge. If Tyto Cartridge is installed correctly, the stage status bar will show ‘Cartridge locked: ready to start measurement’. Workspace: Open a pre-existing workspace by clicking the folder icon in the top toolbar. Select WORKSPACE on the left side of the dialog that pops up and open the desired file. The following saved settings will be recalled: PMT gains, trigger, threshold, analyses pages, and sort gate selection. If creating a new workspace run sample and change PMT gains, trigger, threshold, analyses pages, and sort gate selection until optimal settings achieved (retrieve sample ran for set up if required and reload in pre-sort chamber). Input the processing volume in the experiment tab. Select the trigger channel and cell speed channel base appropriate for experiment. CD362 APC selection of stromal cells proceeded as follows. Tigger BSB (BLUE LASER). Cell speed R1 (RED LASER). Press the play button in the lower right corner of the screen to align the cartridge and start data acquisition. Under the ‘Channels’ tab on the left side of the main screen, if needed, adjust the PMT gains to visualize the desired cell populations in the pre-set plot displays. Press the double arrow icon in the lower right corner of the main screen to start sorting. Monitor and modify Arrival Windows (AW): Under the Tools tab open the Arrival Windows tool. Press Connect and press Sync to synchronize the AW with your channels settings. The trigger channel and the transit channel shall now being displayed in the Detector options tab according to your selection in the channels tab. Press start to initialize data acquisition, the default windows will be visible. Increase the number of displayed events in the Display options tab until you can clearly discriminate a population. Adjust the AW to fully include distribution. Using the Detector1 and Detector 2 tabs change the Minimum and Maximum FWHM values until the window covers the full distribution. To apply the new windows press Program Tables. To save the new AW close the tool. A pop-up window will open, select save. Then save the workspace again. Open FPGA Tool—monitor abort rate—change sort settings or AW settings to reduce abort rate. Sort valve table programmer: For CD362 selection of cord derived stromal cells modification of sort valve timing is necessary due to different flow behavior of the cells. For these cell samples the valve timing can be modified using the sort valve table programmer. Change (min open time=max open time=80 μs). After set-up of the AW open the Sort valve table programmer tool in the Tool tab. Press Sync to synchronize the table with your current settings. Increase Min Open Time and if necessary for cell types Max Open Time, too. Change (min open time=max open time=80 μs). Press program table to transfer the values to the FPGA. Check the correct transfer with the Read Tables button. The actual values will be displayed as dark blue lines. To save the new settings close the tool. A pop-up windows will open, select save. Then save the workspace again. Open Sort Controls table and monitor sort pressure during run. Also monitor sort valve for stick/clumps/blockages. Sort Collection: The sorted cells will be contained in a small volume of MACSQuant Tyto Buffer in the sort chamber (middle luer). Using a gel tip, pipette Tyto Buffer into the sort chamber (100 μl) and retrieve cell suspension and add to labelled Eppendorf tube. Wash the chamber with additional volume (×2 100 μl), and add to recovered cells. For calculation of recovery and yield, the absolute cell number of each fraction must be determined. An aliquot of each fraction should be used to assess the cell concentration either using the MACSQuant flow cytometer.

Enrich Sort Target Cells/Negative Unsorted Cells/Remaining Pre-Sort Fractions: Take a 10 μl sample of HUC Single Cell Suspension and mix with buffer (1:20) in Eppendorf (180 μl). Add Sytox Blue to Eppendorf (1:50) and acquire 180 μl of sample. Record number of viable cells=______. Total Number of cells=Viable Cells×20 (Dilution Factor)×Total Vol: ______. After first Enrichment Sort for CD362+ cells reload sorted cells in 2 mls (1×10e6/2 ml) of Tyto Buffer into a fresh a cartridge (no filter) as previously described. The second run is Purity Sort for CD362+ cells. Monitor sort parameters as previously described. Sort Collection: The sorted cells will be contained in a small volume of MACSQuant Tyto Buffer in the sort chamber (middle luer). Using a gel tip, pipette Tyto Buffer into the sort chamber (100 μl) and retrieve cell suspension and add to labelled Eppendorf tube. Wash the chamber with additional volume (×2 100 μl), and add to recovered cells. For calculation of recovery and yield, the absolute cell number of each fraction must be determined. An aliquot of each fraction should be used to assess the cell concentration either using the MACSQuant flow cytometer. Purity Sort Target Cells/Negative Unsorted Cells/Remaining Pre-Sort Fractions was conducted as follows. Take a 10 μl sample of HUC Single Cell Suspension and mix with buffer (1:20) in Eppendorf (180 μl). Add Sytox Blue to Eppendorf (1:50) and acquire 180 μl of sample. Record number of viable cells=______. Total Number of cells=Viable Cells×20 (Dilution Factor)×Total Vol: ______. Calculate: Initial Purity, Total Target Cells, Enrich Sort Purity, Enrich Sort Target Yield (number & % population), Purity Sort Purity, Purity Sort Target Yield (number & % population), and Overall Yield (number & % population). Culture cells for expansion and for CFU-f analysis.

Analysis of cells isolated using the above procedure was conducted. FIG. 15A shows representative gating strategy for population analysis and CD362+ target identification (population gate→viability gate (sytox blue exclusion)→singlets gate (doublets exclusion)→CD362 APC (target population)). FIG. 15B shows identification of viable CD362+ target stromal cell population (highlighted in red square gate, percentage represent population after gating strategy applied), pre-sort CD362+ (0.38%), post first Tyto sort of CD362+ cells (enrich sort 23%) and final sort of CD362+ cells (purity sort 93%).

Percentage of CD362+ cells following each sort was determined. FIG. 16 shows average percentage population of CD362+ target stromal cell population in pre-sort (0.34±0.15%), enrich sort (14.52±4.1%) and purity sort (80.51±4.3%) (% mean±SEM n=5) after MACSQuant Tyto sorting. (*p≤0.05, ****p≤0.0001, ordinary one-way ANOVA, Holm-Sidak's multiple comparisons test).

Fold enrichment and percent recovery were determined. FIG. 17A shows average fold enrichment of CD362+ target stromal cell population after enrich sort (60±13.63) and purity sort (543.8±235.4) after Tyto sorting, when compared to the pre-sort target population (mean±SEM n=5). FIG. 17B shows average percentage recovery of targets CD362+ population after enrich sort (13.1±3.8%), purity sort (18.58±5.9%) and final the overall yield of target CD362+ stromal cells from the starting population (4.8±1.8%) (% mean±SEM n=4-5).

CFU-F analysis was conducted on the isolated cells. FIG. 18 shows CFU-f analysis of colonies formed from Tyto sorted (purity sort) CD362+ stromal (179.3±35.71 colonies per 1×105 cells plated) compared to standard plastic adherent (PA) unsorted cell fraction (3.4±1.4 colonies per 1×105 cells plated), (mean±SEM, n=3). (**p≤0.01, unpaired student t test).

While preferred embodiments of the present disclosure have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments described herein may be employed. It is intended that the following claims define the scope of the invention and that methods and structures within the scope of these claims and their equivalents be covered thereby.

What is claimed is:

1. A method of treating autoimmune hepatitis in a human in need thereof comprising administering a composition comprising about 2.5×10⁶ stromal stem cells per kilogram body weight to the human, wherein at least 30% of the stromal stem cells in the composition are SDC2+.

2. The method of claim 1, wherein the administering the composition results in reduction of alanine aminotransferase (ALT) levels in the human,

3. The method of claim 1, wherein at least 50% of the stromal stem cells in the composition are SDC2+.

4. The method of claim 1, wherein at least 70% of the stromal stem cells in the composition are SDC2+.

5. The method of claim 1, wherein the human is selected for having a serum alkaline phosphatase (ALP) of at least 1.5× upper limit of normal (ULN) or a serum ALT of at least 1.5×ULN.

6. The method of claim 1, wherein the administering the composition further results in improvement in at least one measure of liver function selected from alkaline phosphatase, aspartate transaminase, albumin, bilirubin, gamma glutamyltransferase, total bile acid, immunoglobulin, and C-reactive protein four weeks after administration of the composition.

7. The method of claim 1, wherein the administering the composition further results in improvement in at least one of osteoporosis, serum cholesterol, xanthomas, absorption of fat soluble vitamins, edema, ascites, hepatic encephalopathy, hypersplenism, hypothyroidism, sicca syndrome, Raynaud's phenomenon, scleroderma, celiac sprue, urinary tract infections, gallstones, jaundice, fatigue, dark urine, pale stool, pain, loss of appetite, and weight loss four weeks after administration of the composition.

8. The method of claim 1, wherein the human maintains healthy liver function for at least 6 months after administration of the composition.
